(19) United States
(12) Patent Application Publication
(10) Pub. No.: US 2024/0016859 A1
(43) Pub. Date: Jan. 18, 2024

(54) MICROENCAPSULATED AND CHROMOSOME INTEGRATED COMPOSITIONS FOR L-DOPA MICROBIOME THERAPY

(71) Applicant: Iowa State University Research Foundation, Inc., Ames, IA (US)

(72) Inventors: Anumantha G. Kanthasamy, Ames, IA (US); Gregory Phillips, Ames, IA (US); Ahmed Abdalla, Ames, IA (US); Nicholas John Backes, Ames, IA (US); Piyush Padhi, Ames, IA (US)

(21) Appl. No.: 18/455,205

(22) Filed: Aug. 24, 2023

Related U.S. Application Data
(62) Division of application No. 17/304,444, filed on Jun. 21, 2021.
(60) Provisional application No. 62/706,098, filed on Jul. 31, 2020.
(60) Provisional application No. 62/706,096, filed on Jul. 31, 2020.

(51) Int. Cl. C12N 1/20; A61P 25/28

(52) U.S. Cl. CPC C12N 1/20; A61P 25/28; C12R 2001/19

(57) ABSTRACT

The present invention generally provides methods and compositions for the treatment of Parkinson's disease, Alzheimer's disease, depression, anxiety, and memory deficits. The invention relates to recombinant microorganisms, particularly gut-colonizing probiotics, modified to produce L-DOPA as well as microcapsules and lyophilized formulations comprising the same.

### CROSS REFERENCE TO RELATED APPLICATIONS

This is a Divisional application of U.S. Ser. No. 17/304,444, filed Jun. 21, 2021, which claims priority to provisional applications U.S. Ser. No. 62/706,096 filed Jul. 31, 2020, and U.S. Ser. No. 62/706,098 filed Jul. 31, 2020, which are incorporated herein by reference in their entireties.

### GOVERNMENT SUPPORT

This invention was made with government support under Grant No. NS112441 awarded by the National Institutes of Health. The Government has certain rights in the invention.

### REFERENCE TO SEQUENCE LISTING SUBMITTED ELECTRONICALLY

The instant application contains a Sequence Listing which has been submitted electronically in XML format and is herein incorporated by reference in its entirety. Said XML copy, created on Aug. 23, 2023, is named “P13188US03_SequenceListing.xml” and is 5,119 bytes in size.

### TECHNICAL FIELD

This invention relates generally to compositions comprising a recombinant microbial cell, specifically to a probiotic strain engineered to produce L-DOPA, and methods of using the same to provide L-DOPA in a sustained manner for treatment of Parkinson's disease and other Parkinsonian disorders. Uses of the compositions for treatment of Alzheimer's disease, depression, anxiety, learning and memory deficits, and other related mood disorders are also disclosed.

### BACKGROUND

More than 10 million people worldwide, and one million Americans have Parkinson's disease (PD), and approximately 50,000 new cases are diagnosed each year, with the incidence among aging population exceeding that for other younger segments of the US population. In addition, Parkinsonian disorders including progressive supranuclear palsy (PSP) and multiple system atrophy (MSA) have overlapping neurological deficits and clinical pathology with PD. The cardinal pathology of PD is progressive neurodegeneration of dopamine-producing neurons in substantia nigra, contributing to dopamine deficiency that manifest in severe motor symptoms including rigidity, bradykinesia, tremors, and postural instability. The non-motor prodromal symptoms such as constipation, anosmia, sleep disturbances, depression, anxiety and memory deficits are also well documented in PD and Parkinsonism. Many patients have both PD and Alzheimer's Disease (AD) pathologies and symptoms. The amino acid L-DOPA is the precursor to the neurotransmitter dopamine, and has been used for the treatment of a variety of neurological disorders including PD. The discovery of dopamine replacement therapy with Levodopa (L-DOPA) for PD represents one of the most remarkable success stories in the history of medicine. For decades, L-DOPA is the drug most often prescribed because it's unparalleled symptomatic relief to PD patients. Unfortunately, L-DOPA gold standard therapy met inherent side effects commonly referred to L-DOPA induced dyskinesia (LID). Although neurochemical basis of LID is not completely understood, dopamine receptor sensitization due to pulsated delivery of L-DOPA in form of 100-500 mg tablet 2 to 3 times day is considered to be major reason for the LID. The clinical diagnosis of L-DOPA drug fluctuations include peak dose, off period dystonia, and diphasic dyskinesia. In the face of antiparkinsonian treatment, the lack of effective treatment to control LID remains by far the most challenging problem.

Therefore, it is an object of the present invention to provide methods and compositions to deliver L-DOPA in a sustained manner thereby avoiding pulsated delivery and the L-DOPA induced dyskinesia associated with the standard treatment regimen. Other objects will become apparent from the description of the invention which follows.

### SUMMARY

The present invention provides methods and compositions comprising a recombinant microbial cell capable of producing L-DOPA, for use in the treatment of Parkinson's disease, Alzheimer's disease, depression, anxiety, and memory deficits. In some embodiments, the recombinant microbial cell colonizes the gut of the subject in need of treatment, thereby providing L-DOPA in a sustained manner.

In some embodiments, the recombinant microbial cell is a probiotic. In an exemplary embodiment, the probiotic is E. coli Nissle 1917. In some embodiments, the recombinant microbial cell capable of producing L-DOPA comprises a hpaB nucleotide sequence and a hpaC nucleotide sequence. In some embodiments, the hpaBC nucleotide sequence comprises the sequence set forth in SEQ ID NO: 1. The hpaB and hpaC nucleotide sequence may be stably integrated into the genome of the recombinant microbial cell or contained in a plasmid.

Microencapsulated compositions comprising the recombinant microbial cells are disclosed. The microcapsules include a core component comprising the recombinant microbial cell and a coating material surrounding the core component. In some embodiments, the core component further comprises an aromatic amino acid- or DOPA-decarboxylase inhibitor such as carbidopa or benserazide. In some embodiments, the coating material comprises polymer. In another embodiment, the coating material comprises alginic acid or an alginate.

In another embodiment, the composition is lyophilized and may further comprise a cryoprotectant. In yet another embodiment, the composition further comprises a pharmaceutically acceptable carrier.

The disclosure provides a method for providing a subject in need thereof with a treatment for Parkinson's disease comprising administering to the subject an effective amount of a composition comprising a recombinant microbial cell capable of producing L-DOPA. In another embodiment, methods of treating Alzheimer's disease, depression and/or anxiety, memory deficits, and improving motivation to do difficult tasks are provided. The method comprises administering to the subject in need thereof an effective amount of a composition comprising a recombinant microbial cell capable of producing L-DOPA. In some embodiments, the composition is administered orally. In some embodiments, the composition is administered on alternate days. In certain embodiments, the subject is a mammal. In a preferred embodiment, the mammal is a human.

While multiple embodiments are disclosed, still other embodiments of the present invention will become apparent to those skilled in the art from the following detailed description, which shows and describes illustrative embodiments of the invention. Accordingly, the figures and detailed description are to be regarded as illustrative in nature and not restrictive.

### BRIEF DESCRIPTION OF THE FIGURES

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1 shows plasmid constructs used for expression of hpaBC in EcN. Synthetic hpaBC genes were cloned into the pRHAM vector yielding the plasmid shown. The rha promoter and operator were also replaced with three individual constitutive promoters (P1, P2, P3). Along with the coding regions for hpaB and hpaC, additional plasmid features include: kan, kanamycin resistance; rop, control of plasmid copy number; ColEIori, origin of replication.

FIG. 2 shows a summary of the ROPE integration system. The hpaBC genes cloned into the pROPE plasmid are liberated as a linear DNA fragment by expression of the I-SceI nuclease (expressed from the helper plasmid pSLTS). The homologous DNA arms (H1, H2) allow repair of a chromosomal DNA break also initiated by I-SceI by recombination resulting in integration of the hpaBC genes into the lac (lactose) operon of EcN.

FIG. 3 is a diagram of genetic configuration of the EcN^{rha}_{L-DOPA} systems.

FIGS. 4A-B show the dose-dependent increase in total L-DOPA due to concentration effect of rhamnose inducer in EcN_{LDOPA}⁴ in vitro. FIG. 4A shows L-DOPA produced in vitro. FIG. 4B shows norepinephrine (NE) produced in vitro.

FIGS. 5A-D show orally administered EcN_{LDOPA}^{GEN 2/4} is efficacious in attenuating spatial learning and memory deficits in MitoPark Mice.

FIGS. 6A-C show orally administered EcN_{LDOPA}^{GEN 2/4} is efficacious in attenuating locomotor deficits in MitoPark Mice. FIGS. 6A, 6B, and 6C shows horizontal, vertical and ambulatory activity plots, respectively.

FIG. 7 shows orally administered EcN_{LDOPA}⁴ increases plasma L-DOPA in mouse mice.

FIGS. 8A-D show EcN_{LDOPA}² and EcN_{LDOPA}⁴ viability and release kinetics of L-DOPA from the lyophilized formulation.

FIGS. 9A-B show oral administration of the liquid formulation EcN_{LDOPA}⁴. FIG. 9A shows EcN_{LDOPA}⁴ increases plasma L-DOPA levels in dogs. FIG. 9B shows EcN_{LDOPA}⁴ increases CSF L-DOPA in dogs.

FIG. 10 is a schematic of microencapsulation.

FIG. 11 shows the release kinetics of Escherichia coli Nissle 1917 (EcN) genetically engineered to produce L-DOPA (EcN_{LDOPA}) in calcium-alginate microcapsule.

FIG. 12 shows the L-DOPA release kinetics from calcium-alginate EcN_{LDOPA} microcapsules.

FIG. 13 shows the release kinetics of L-DOPA from respective number of EcN_{LDOPA} calcium-alginate microcapsules.

FIG. 14 shows the release kinetics of benserazide from calcium-alginate microcapsules.

FIG. 15 show generations of EcN_{LDOPA} engineered to develop lead therapeutic.

FIGS. 16A-B show EcN_{LDOPA} significantly increases Striatal DA in C57BL/6 following single administration.

FIG. 17 shows EcN_{LDOPA} significantly rescues locomotor deficits in MitoPark animal model of PD.

FIG. 18 shows EcN_{LDOPA} moderately improves depressive-like behavior in MitoPark animal model of PD.

FIG. 19 shows chronic administration of EcN_{LDOPA} ensures stable colonization profile in MitoPark animal model of PD.

FIG. 20 shows plasma L-DOPA chronic dose pharmacokinetic profile in MitoPark animal model of PD.

FIGS. 21A-C show EcN_{LDOPA} significantly improves dopamine and norepinephrine neurochemical profile in MitoPark animal model of PD following chronic administration. *p<0.05, **p<0.005, ****p<0.0001.

FIG. 22 shows significant levels of EcN_{L-DOPA} were detected in fecal samples of APP-KI rodents and their respective age-matched littermate controls.

FIG. 23 shows EcN_{L-DOPA} improves hippocampal Dopamine levels in APP-KI rodents.

FIG. 24 shows EcN_{L-DOPA} improves pre-frontal cortex norepinephrine levels in APP-KI rodents.

FIG. 25 shows EcN_{L-DOPA} effectively colonized in the gut of LC-lesioned Tg 344 AD rats.

FIG. 26 shows EcN_{L-DOPA} increases plasma L-DOPA in LC-Lesioned Tg344 rat model. Tukey's multiple unpaired T-test **p<0.006, ***p<0.002.

FIG. 27 shows EcN_{L-DOPA} dramatically increased pre-frontal cortical NE levels in LC-Lesioned Tg344 rat model. Tukey's multiple unpaired t-test ****p<0.0001.

### DETAILED DESCRIPTION

The present invention provides methods and compositions comprising a recombinant microbial cell capable of producing L-DOPA. The recombinant microbial cell colonizes the gut of the subject in need of treatment and provides L-DOPA in a sustained manner to avoid the development of Levodopa-induced dyskinesia (LID).

So that the present invention may be more readily understood, certain terms are first defined. Unless defined otherwise, all technical and scientific terms used herein have the same meaning as commonly understood by one of ordinary skill in the art to which embodiments of the invention pertain. Many methods and materials similar, modified, or equivalent to those described herein can be used in the practice of the embodiments of the present invention without undue experimentation, the preferred materials and methods are described herein. In describing and claiming the embodiments of the present invention, the following terminology will be used in accordance with the definitions set out below.

The singular terms “a”, “an”, and “the” include plural referents unless context clearly indicates otherwise. Similarly, the word “or” is intended to include “and” unless the context clearly indicate otherwise. The word “or” means any one member of a particular list and also includes any combination of members of that list.

Numeric ranges recited within the specification, including ranges of “greater than,” “at least”, or “less than” a numeric value, are inclusive of the numbers defining the range and include each integer within the defined range. For example, when a range of “1 to 5” is recited, the recited range should be construed as including ranges “1 to 4”, “1 to 3”, “1-2”, “1-2 & 4-5”, “1-3 & 5”, and the like.

The term “about” as used herein, refers to variation in the numerical quantity that can occur, for example, through typical measuring techniques and equipment, with respect to any quantifiable variable, including, but not limited to, mass, volume, time, distance, wave length, frequency, voltage, current, and electromagnetic field. Further, given solid and liquid handling procedures used in the real world, there is certain inadvertent error and variation that is likely through differences in the manufacture, source, or purity of the ingredients used to make the compositions or carry out the methods and the like. The term “about” also encompasses amounts that differ due to different equilibrium conditions for a composition resulting from a particular initial mixture. The term “about” also encompasses these variations. Whether or not modified by the term “about,” the claims include equivalents to the quantities.

As used herein, the terms “microbe”, “microbial cell”, or “microorganism” refer to an organism of microscopic, submicroscopic, or ultramicroscopic size that typically consists of a single cell. Examples of microorganisms include bacteria, viruses, fungi, certain algae, and protozoa. The term “microbial” indicates pertaining to, or characteristic of a microorganism.

The term “microbiome”, as used herein, refers to a population of microorganisms from a particular environment, including the environment of the body or a part of the body. The term is interchangeably used to address the population of microorganisms itself (sometimes referred to as the microbiota), as well as the collective genomes of the microorganisms that reside in the particular environment. The term “environment,” as used herein, refers to all surrounding circumstances, conditions, or influences to which a population of microorganisms is exposed. The term is intended to include environments in a subject, such as a human and/or animal subject.

“Probiotic” is used to refer to live, non-pathogenic microorganisms, e.g., bacteria, which can confer health benefits to a host organism that contains an appropriate amount of the microorganism.

“Gut” refers to the organs, glands, tracts, and systems that are responsible for the transfer and digestion of food, absorption of nutrients, and excretion of waste. In humans, the gut comprises the gastrointestinal tract, which starts at the mouth and ends at the anus, and additionally comprises the esophagus, stomach, small intestine, and large intestine. The gut also comprises accessory organs and glands, such as the spleen, liver, gallbladder, and pancreas. The upper gastrointestinal tract comprises the esophagus, stomach, and duodenum of the small intestine. The lower gastrointestinal tract comprises the remainder of the small intestine, i.e., the jejunum and ileum, and all of the large intestine, i.e., the cecum, colon, rectum, and anal canal. Bacteria can be found throughout the gut, e.g., in the gastrointestinal tract, and particularly in the intestines.

As used herein, “nucleic acid” includes reference to a deoxyribonucleotide or ribonucleotide polymer in either single- or double-stranded form, and unless otherwise limited, encompasses known analogues having the essential nature of natural nucleotides in that they hybridize to single-stranded nucleic acids in a manner similar to naturally occurring nucleotides. Unless otherwise indicated, the term includes reference to the specified sequence as well as the complementary sequence thereof.

As used herein, the terms “peptide”, “polypeptide”, and “protein” will be used interchangeably to refer to a chain of amino acids each of which is joined to the next amino acid by a peptide bond. In one aspect, this term also includes post translational modifications of the polypeptide, for example, glycosylations, acetylations, phosphorylations and the like. Included within the definition are, for example, peptides containing one or more analogues of an amino acid or labeled amino acids and peptidomimetics.

The terms “residue” or “amino acid residue” or “amino acid” are used interchangeably herein to refer to an amino acid that is incorporated into a protein, polypeptide, or peptide (collectively “protein”). The amino acid may be a naturally occurring amino acid and, unless otherwise limited, may encompass known analogs of natural amino acids that can function in a similar manner as naturally occurring amino acids.

“Regulatory elements” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory elements may include, but are not limited to, promoters, translation leader sequences, introns, and polyadenylation recognition sequences. Regulatory elements present on a recombinant DNA construct that is introduced into a cell can be endogenous to the cell, or they can be heterologous with respect to the cell. The terms “regulatory element” and “regulatory sequence” are used interchangeably herein.

As used herein “promoter” includes reference to a region of DNA upstream from the start of transcription and involved in recognition and binding of RNA polymerase and other proteins to initiate transcription. An “inducible” or “repressible” promoter is a promoter which is under environmental control. Examples of environmental conditions that may affect transcription by inducible promoters include anaerobic conditions or the presence of light. Tissue specific, tissue preferred, cell type specific, and inducible promoters constitute the class of “non-constitutive” promoters. A “constitutive” promoter is a promoter which is active under most environmental conditions.

“Operably linked” refers to the association of nucleic acid fragments in a single fragment so that the function of one is regulated by the other. For example, a promoter is operably linked with a nucleic acid fragment when it is capable of regulating the transcription of that nucleic acid fragment.

As used herein, “vector” refers to a DNA or RNA molecule (such as a plasmid, linear piece of DNA, cosmid, bacteriophage, yeast artificial chromosome, or virus, among others) that carries nucleic acid sequences into a host cell. The vector or a portion of it can be inserted into the genome of the host cell.

As used herein the term “codon-optimized” refers to the modification of codons in the gene or coding regions of a nucleic acid molecule to reflect the typical codon usage of the host organism without altering the polypeptide encoded by the nucleic acid molecule. Such optimization includes replacing at least one, or more than one, or a significant number, of codons with one or more codons that are more frequently used in the genes of the host organism. A “codon-optimized sequence” refers to a sequence, which was modified from an existing coding sequence, or designed, for example, to improve translation in an expression host cell or organism of a transcript RNA molecule transcribed from the coding sequence, or to improve transcription of a coding sequence. Codon optimization includes, but is not limited to, processes including selecting codons for the coding sequence to suit the codon preference of the expression host organism. Many organisms display a bias or preference for use of particular codons to code for insertion of a particular amino acid in a growing polypeptide chain. Codon preference or codon bias, differences in codon usage between organisms, is allowed by the degeneracy of the genetic code, and is well documented among many organisms. Codon bias often correlates with the efficiency of translation of messenger RNA (mRNA), which is in turn believed to be dependent on, inter alia, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

The term “introduced” in the context of inserting a nucleic acid into a cell, means “transfection” or “transformation” or “transduction” and includes reference to the incorporation of a nucleic acid into a eukaryotic or prokaryotic cell where the nucleic acid may be incorporated into the genome of the cell (e.g., chromosome, plasmid, plastid or mitochondrial DNA), converted into an autonomous replicon, or transiently expressed (e.g., transfected mRNA).

As used herein, “transformation” refers to a process of introducing an exogenous nucleic acid molecule (e.g, a vector, a recombinant DNA molecule) into a host cell. Transformation typically achieves a genetic modification of the cell. The introduced nucleic acid may integrate into a chromosome of a cell, or may replicate autonomously. A cell that has undergone transformation, or a descendant of such a cell, is “transformed” and is a “recombinant” cell. Recombinant cells are modified cells as described herein. Cells herein may be transformed with, for example, one or more of a vector, a plasmid or a linear piece (eg., a linear piece of DNA created by linearizing a vector) of DNA. The plasmid or linear piece of DNA may or may not comprise a selectable or screenable marker.

As used herein “recombinant” includes reference to a cell or vector, that has been modified by the introduction of a heterologous nucleic acid or that the cell is derived from a cell so modified. Thus, for example, recombinant cells express genes that are not found in identical form within the native (non-recombinant) form of the cell or express native genes that are otherwise abnormally expressed, under-expressed or not expressed at all as a result of deliberate human intervention. The term “recombinant” as used herein does not encompass the alteration of the cell or vector by naturally occurring events (e.g., spontaneous mutation, natural transformation/transduction/transposition) such as those occurring without deliberate human intervention.

As used herein, the term “treating” means ameliorating, improving or remedying a disease, disorder, or symptom of a disease or condition. For example, with respect to Parkinson's disease, treatment may be measured by quantitatively or qualitatively to determine the presence/absence of the disease, or its progression or regression using, for example, symptoms associated with the disease or clinical indications associated with the pathology.

As used herein, the term “subject”, “individual”, or “patient” refers to any organism upon which embodiments of the invention may be used or administered, e.g., for experimental, diagnostic, prophylactic, and/or therapeutic purposes. In some embodiments, a subject is a mammal, e.g., a human or non-human primate (e.g., an ape, monkey, orangutan, or chimpanzee), a dog, cat, guinea pig, rabbit, rat, mouse, horse, cattle, or cow.

As used herein a “pharmaceutical composition” refers to a preparation of recombinant microbial cells of the invention with other components such as a pharmaceutically acceptable carrier and/or excipient.

As used herein, the term “pharmaceutically acceptable carrier” refers to any carrier, diluent, excipient, wetting agent, buffering agent, suspending agent, lubricating agent, adjuvant, vehicle, delivery system, emulsifier, disintegrant, absorbent, preservative, surfactant, colorant, flavorant, or sweetener, preferably non-toxic, that would be suitable for use in a pharmaceutical composition. The compositions of the present invention may be administered in the form of a pharmaceutical composition with a pharmaceutically acceptable carrier. Pharmaceutically acceptable carriers may be chosen to permit oral administration or administration by any other known route.

The term “excipient” refers to an inert substance added to a pharmaceutical composition to further facilitate administration of an active ingredient. Examples include, but are not limited to, calcium bicarbonate, calcium phosphate, various sugars and types of starch, cellulose derivatives, gelatin, vegetable oils, polyethylene glycols, and surfactants, including, for example, polysorbate 20.

As used herein, the terms “pharmaceutically effective” or “therapeutically effective” shall mean an amount of a composition that is sufficient to show a meaningful patient benefit, i.e., treatment, prevention, amelioration, or a decrease in the frequency of the condition or symptom being treated.

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject.

As used herein, the term “combination therapy” refers to the administration of the recombinant microbial cell with an at least one additional pharmaceutical or medicinal agent (e.g., an anxiolytic agent), either sequentially or simultaneously.

### Recombinant Microbial Cells

The recombinant cell according to the invention may be constructed from any suitable host cell. The host cell may be an unmodified cell or may already be genetically modified. In one embodiment, the recombinant microbial cell is recombinant gut-colonizing microbial cell. The cell may be a prokaryotic cell or a eukaryotic cell. In one embodiment, the cell is a prokaryotic cell.

In one embodiment, the recombinant microbial cell is a nonpathogenic bacterial cell. In some embodiments, the recombinant microbial cell is a commensal bacterial cell. In some embodiments, the recombinant microbial cell is a yeast cell. In some embodiments, the recombinant microbial cell is a naturally pathogenic microbial cell that is modified or mutated to reduce or eliminate pathogenicity.

The recombinant microbial cell may be a probiotic. Some species, strains, and/or subtypes of non-pathogenic microorganisms are currently recognized as probiotic. Examples of probiotic microorganisms include, but are not limited to, Bifidobacteria, Escherichia coli, Lactobacillus, and Saccharomyces, e.g., Bifidobacterium bifidum, Enterococcus faecium, Escherichia coli strain Nissle 1917, Lactobacillus acidophilus, Lactobacillus bulgaricus, Lactobacillus paracasei, Lactobacillus plantarum, and Saccharomyces boulardii (Dinleyici et al., 2014; U.S. Pat. Nos. 5,589,168; 6,203,797; 6,835,376).

Examples of probiotic bacteria include, but are not limited to, specific probiotic strains of Lactobacillus, Bifidobacterium, Lactococcus, Enterococcus, Streptococcus, Pediococcus, Leuconostoc, Bacillus, or Escherichia coli.

In some embodiments, a probiotic Lactobacillus may include, without limitation, a Lactobacillus reuteri, Lactobacillus plantarum, Lactobacillus casei (such as Lactobacillus casei Shirota), Lactobacillus salivarius, Lactobacillus paracasei, Lactobacillus lactis, Lactobacillus acidophilus, Lactobacillus sakei, Lactobacillus brevis, Lactobacillus buchneri, Lactobacillus fermentum, Lactobacillus delbrueckii, Lactobacillus delbrueckii subsp. bulgaricus, Lactobacillus helveticus, Lactobacillus garvieae, Lactobacillus acetotolerans, Lactobacillus agilis, Lactobacillus algidus, Lactobacillus alimentarius, Lactobacillus amylolyticus, Lactobacillus amylophilus, Lactobacillus amylovorus, Lactobacillus animalis, Lactobacillus aviarus, Lactobacillus bifermentans, Lactobacillus bulgaricus, Lactobacillus camis, Lactobacillus caternaformis, Lactobacillus cellobiosis, Lactobacillus collinoides, Lactobacillus confuses, Lactobacillus coryniformis, Lactobacillus crispatus, Lactobacillus curvatus, Lactobacillus divergens, Lactobacillus farciminis, Lactobacillus fructivorans, Lactobacillus fructosus, Lactobacillus gallinarum, Lactobacillus gasseri, Lactobacillus graminis, Lactobacillus haiotoierans, Lactobacillus hamster, Lactobacillus heterohiochii, Lactobacillus hilgardii, Lactobacillus homohiochii, Lactobacillus iners, Lactobacillus intestinalis, Lactobacillus jensenii, Lactobacillus johnsonii, Lactobacillus kandleri, Lactobacillus kefiri, Lactobacillus kefuranofaciens, Lactobacillus kefirgranum, Lactobacillus kunkeei, Lactobacillus leichmannii, Lactobacillus llndnerl, Lactobacillus malefermentans, Lactobacillus mall, Lactobacillus maltaromicus, Lactobacillus manihotivorans, Lactobacillus minor, Lactobacillus minutus, Lactobacillus mucosae, Lactobacillus murinus, Lactobacillus nagelii, Lactobacillus oris, Lactobacillus panis, Lactobacillus parabuchneri, Lactobacillus paracasei, Lactobacillus parakefiri, Lactobacillus paralimentarius, Lactobacillus paraplantarum, Lactobacillus pentosus, Lactobacillus perolens, Lactobacillus piscicola, Lactobacillus plantarum, Lactobacillus pontis, Lactobacillus rhamnosus, Lactobacillus rhamnosus GG, Lactobacillus rimae, Lactobacillus rogosae, Lactobacillus ruminis, Lactobacillus sanfranciscensis, Lactobacillus sharpeae, Lactobacillus suebicus, Lactobacillus trichodes, Lactobacillus uli, Lactobacillus vaccinostercus, Lactobacillus vaginalis, Lactobacillus viridescens, Lactobacillus vitulinus, Lactobacillus xylosus, Lactobacillus yamanashiensis, or a Lactobacillus zeae.

In some embodiments, a probiotic Escherichia coli may be E. coli Nissle 1917. As used herein, the term “Escherichia” refers to a genus of Gram-negative, non-spore forming, facultatively anaerobic, rod-shaped bacteria from the family Enterobacteriaceae. The genus Escherichia include various species, such as Escherichia coli. The terms “Escherichia coli Nissle 1917” or “EcN” as used herein refer to a non-pathogenic Gram-negative probiotic bacteria Escherichia coli strain that is capable of colonizing the human gut. In an exemplary embodiment, the probiotic is the Escherichia coli strain Nissle 1917.

Escherichia coli Nissle 1917 has evolved into one of the best characterized probiotics (Ukena et al., 2007). The strain is characterized by its complete harmlessness (Schultz, 2008), and has GRAS (generally recognized as safe) status (Reister et al., 2014). E. coli strain Nissle 1917 lacks defined virulence factors such as alpha-hemolysin, other toxins, and mannose-resistant hemagglutinating adhesins (Blum et al. Infection. 23(4):234-236 (1996)), P-fimbrial adhesins, and the semi-rough lipopolysaccharide phenotype and expresses fitness factors such as microcins, ferritins, six different iron uptake systems, adhesins, and proteases, which support its survival and successful colonization of the human gut (Grozdanov et al. J Bacteriol. 186(16): 5432-5441 (2004)). As early as in 1917, E. coli Nissle was packaged into medicinal capsules, called MUTAFLOR®, for therapeutic use. E. coli Nissle has since been used to treat ulcerative colitis in humans in vivo (Rembacken et al., 1999), to treat inflammatory bowel disease, Crohn's disease, and pouchitis in humans in vivo (Schultz, 2008), and to inhibit enteroinvasive Salmonella, Legionella, Yersinia, and Shigella in vitro (Altenhoefer et al., 2004). It is commonly accepted that E. coli Nissle's therapeutic efficacy and safety have convincingly been proven (Ukena et al., 2007).

Examples of Escherichia coli Nissle 1917 bacteria include those available as DSM 6601 from the German Collection for Microorganisms in Braunschweig, Germany or commercially as the active component in MUTAFLOR® (Ardeypharm GmbH, Herdecke, Germany).

In some embodiments, a probiotic Bifidobacterium may be Bifidobacterium infantis, Bifidobacterium adolescentis, Bifidobacterium animalis subsp animalis, Bifidobacterium longum, Bifidobacterium fidobacterium breve, Bifidobacterium bifidum, Bifidobacterium animalis subsp. lactis or Bifidobacterium lactis, such as Bifidobacterium lactis DN-173 010.

In some embodiments, a probiotic Bacillus may be Bacillus coagulans. In some embodiments, a probiotic Lactococcus may be Lactococcus lactis subsp. Lactis such as Lactococcus lactis subsp. lactis CV56. In some embodiments, a probiotic Enterococcus may be Enterococcus durans. In some embodiments, a probiotic Streptococcus may be Streptococcus thermophilus.

In some embodiments, the probiotic bacterium may be an auxotrophic strain designed, for example, to limit its survival outside of the human or animal intestine, using standard techniques.

The probiotic may be a variant or a mutant strain of bacterium (Arthur et al., 2012; Cuevas-Ramos et al., 2010; Olier et al., 2012; Nougayrede et al., 2006). Non-pathogenic bacteria may be genetically engineered to enhance or improve desired biological properties, e.g., survivability. Non-pathogenic bacteria may be genetically engineered to provide probiotic properties. Probiotic cells may be genetically engineered to enhance or improve probiotic properties, e.g., enhance gut colonization.

Bacterial strains can be readily obtained using standard methods known in the art. For example, a commensal bacterium such as Escherichia coli Nissle 1917 can be obtained from a commercial preparation of the probiotic MUTAFLOR®. Bacteria can be cultured using standard methods known in the art.

One of ordinary skill in the art would appreciate that the genetic modifications disclosed herein may be modified and adapted for other species, strains, and subtypes of bacteria or other microorganisms.

The recombinant microbial cell may be capable of producing L-DOPA and colonizing the gut of a subject. In some embodiments, the recombinant cell comprises a nucleic acid molecule encoding 4-hydroxyphenylacetate 3-monooxygenase (hpaB) and its FAD reductase (hpaC) for the biosynthesis of L-DOPA from L-tyrosine. In some embodiments, the nucleic acid molecule has at least 80%, 85%, 90%, 95%, 96%, 97%, 98%, 99%, or 100% sequence identity to SEQ ID NO: 1. In some embodiments, the nucleic acid molecule comprises or consists of the nucleic acid sequence of SEQ ID NO: 1. Additionally, other hpaB and hpaC nucleic acid sequences may be identified through databases such as Genbank. The hpaB and hpaC nucleotide sequence may be contained in a plasmid of the recombinant microbial cell. The hpaB and hpaC nucleotide sequence may be stably integrated into the genome of the recombinant microbial cell to avoid antibiotic selection requirements and potential loss of recombinant plasmids.

The hpaBC nucleotide sequence may be operably linked to a promoter sequence. In some embodiments, the promoter is a constitutive promoter or an inducible promoter. Inducible promoters allow the transcription to be turned on and modulated by the addition of an inducer. In some embodiments, the inducer may be administered with the composition. In some embodiments, the composition is preincubated with the inducer prior to administration. The inducer can be metabolites such as a sugar or environmental conditions such as hypoxia, temperature, or pH. In an exemplary embodiment, the promoter is a rhamnose-inducible promoter. Since it is a non-metabolizable inert sugar, rhamnose may be used clinically. In an embodiment, the rhamnose-inducible promoter is the rhaB promoter of the E. coli rhaBAD operon (SEQ ID NO: 2).

A nucleic acid may be introduced into a cell by conventional methods, such as, for example, electroporation (see, e.g., Heiser W. C. Transcription Factor Protocols: Methods in Molecular Biology™ 2000; 130: 117-134), chemical (e.g., calcium phosphate or lipid) transfection (see, e.g., Lewis W. H., et al., Somatic Cell Genet. 1980 May; 6(3): 333-47; Chen C., et al., Mol Cell Biol. 1987 August; 7(8): 2745-2752), fusion with bacterial protoplasts containing recombinant plasmids (see, e.g., Schaffner W. Proc Natl Acad Sci USA. 1980 April; 77(4): 2163-7), transduction, conjugation, or microinjection of purified DNA directly into the nucleus of the cell (see, e.g., Capecchi M. R. Cell. 1980 November; 22(2 Pt 2): 479-88).

In one aspect, the present disclosure is directed towards recombinant Escherichia coli Nissle 1917 (EcN) cell, or a variant thereof, transformed with a nucleic acid molecule containing one or more genes involved in the biosynthesis of L-DOPA. As demonstrated in the Examples, the inventors have determined that EcN cells transformed with plasmids expressing hpaB and hpaC are useful for the recombinant production of L-DOPA.

Accordingly, in one embodiment there is provided a recombinant EcN cell or variant thereof comprising hpaB and hpaC (SEQ ID NO: 1). Optionally, the recombinant EcN cell comprises one or more genes with sequence similarity to hpaB and hpaC (SEQ ID NO: 1). For example, in one embodiment, the cell comprises one or more nucleic acid sequences with at least 70%, 75%, 80%, 85%, 90%, 95%, 96%, 97%, 98%, 99% or 100% sequence identity to SEQ ID NO: 1.

Sequence identity can be determined according to sequence alignment methods known in the art. Examples of these methods include computational methods such as those that make use of the BLAST algorithm, available online from the National Center for Biotechnology Information. Sequence identity is most preferably assessed by the algorithm of BLAST version 2.1 advanced search. BLAST is a series of programs that are available, for example, online from the National Institutes of Health. References to BLAST searches are: Altschul, S. F., Gish, W., Miller, W., Myers, E. W. & Lipman, D. J. (1990) “Basic local alignment search tool.” J. Mol. Biol. 215:403410; Gish, W. & States, D. J. (1993) “Identification of protein coding regions by database similarity search.” Nature Genet. 3:266272; Madden, T. L., Tatusov, R. L. & Zhang, J. (1996) “Applications of network BLAST server” Meth. Enzymol. 266:131_141; Altschul, S. F., Madden, T. L., Schaffer, A. A., Zhang, J., Zhang, Z., Miller, W. & Lipman, D. J. (1997) “Gapped BLAST and PSI BLAST: a new generation of protein database search programs.” Nucleic Acids Res. Zhang, J. & Madden, T. L. (1997) “PowerBLAST: A new network BLAST application for interactive or automated sequence analysis and annotation.” Genome Res. 7:649656.

Percent sequence identity or homology between two sequences is determined by comparing a position in the first sequence with a corresponding position in the second sequence. When the compared positions are occupied by the same nucleotide or amino acid, as the case may be, the two sequences are conserved at that position. The degree of conservation between two sequences is often expressed as a percentage representing the ratio of the number of matching positions in the two sequences to the total number of positions compared. Two nucleic acid, or two polypeptide sequences are substantially homologous to each other when the sequences exhibit at least about 70%-75%, preferably 80%-82%, more preferably 85%-90%, even more preferably 92%, still more preferably 95%, and most preferably 98% sequence identity over a defined length of the molecules, as determined using the methods above.

Nucleic acid hybridization may also be used to identify substantially similar nucleic acid molecules to those reported herein. The present nucleic acid molecules described herein may be used to identify genes encoding substantially similar polypeptides/proteins expected to have similar function. Nucleic acid hybridization may be conducted under stringent conditions. Substantially similar sequences are defined by their ability to hybridize, under the following stringent conditions (0.1×SSC, 0.1% SDS, 65° C. and washed with 2×SSC, 0.1% SDS followed by 0.1×SSC, 0.1% SDS, 65° C.).

### Microencapsulation

The recombinant microbial cell may be microencapsulated. Microencapsulation of the recombinant microbial cell can protect against low pH, a high bile-salt concentration, and high temperatures.

The microcapsules may be of any size or shape. Basic geometrical shapes may be, for example, spheres, rods, cylinders, cubes, cuboids, prism, pyramids, cones, truncated cones and truncated pyramids. The microcapsules may be of regular shape or may have be irregular in shape. The surface of the microcapsule may be smooth, uneven, or jagged. They may be amorphous, spherical, or acicular in shape, depending on the respective method of production. In a single dosage that includes microcapsules, the microcapsules may be of uniform size and shape, or may be of variables sizes and shapes.

The microcapsules may be of any size. For example, the maximum diameter of the microcapsule may be about 10 nm, 100 nm, 1 μm, 10 μm, 50 μm, 100 μm, 200 μm, 300 μm, 400 μm, 500 μm, 600 μm, 700 μm, 800 μm, 900 μm, 1 mm, 1.5 mm, 2.0 mm, 2.5 mm, 3.0 mm, 3.5 mm, 4.0 mm, 4.5 mm, 5.0 mm, 5.5 mm, 6.0 mm, 6.5 mm, 7.0 mm, 7.5 mm, 8.0 mm, 8.5 mm, 9.0 mm, 9.5 mm, 1.0 cm or greater, or any range of maximum diameters derivable within the aforementioned maximum diameters. For example, the maximum diameter of the microcapsule may range from about 10 nm to about 1.0 cm. In some embodiments, the mean diameter ranges from about 100 μm to about 1 mm. In some embodiments, the mean diameter ranges from about 100 μm to about 0.1 mm.

The microcapsule may comprise at least 5%, at least 10%, at least 20%, at least 30%, at least 40%, at least 50%, at least 60%, at least 70%, at least 80%, at least 90%, at least 95%, at least 99% or more of the recombinant microbial cells by weight.

The microcapsules may be formed using any method known to those of ordinary skill in the art. Methods for preparing microcapsules are discussed in the following U.S. Patent Application Pub. Nos.: 20080022965, 20080193653, 20070138673; 20070082829; 20060234053, 20060121122, 20050113282, 20040121155, 20040074089, and 20020009473, and the following U.S. Pat. Nos. 7,576,903, 7,037,582, 6,936,644, 6,653,256, 6,592,916, 6,486,099, 4,460,722, each of which is herein specifically incorporated by reference.

The core as used herein refers to that portion of the microcapsule that includes the recombinant microbial cell, where the recombinant microbial cell is encased in a coating material. The coating material may comprise a crosslinked nanopolymer. Some examples of coating materials include cellulose acetate pthalate, methyl acrylate-methacrylic acid copolymers, cellulose acetate succinate, hydroxy propyl methyl cellulose phthalate, hydroxy propyl methyl cellulose acetate succinate, polyvinyl acetate phthalate, methyl methacrylate-methacrylic acid copolymers, alginates, and stearic acid. The coating may include suitable hydrophilic gelling polymers including but not limited to cellulosic polymers, such as methylcellulose, carboxymethylcellulose, hydroxypropylcellulose, hydroxypropylmethylcellulose, hydroxyethylcellulose, and the like; vinyl polymers, such as polyvinylpyrrolidone, polyvinyl alcohol, polyanhydride, and the like; acrylic polymers and copolymers, such as acrylic acid polymer, methacrylic acid copolymers, ethyl acrylate-methyl methacrylate copolymers, natural and synthetic gums, such as guar gum, arabic gum, xanthan gum, gelatin, collagen, proteins, polysaccharides, such as pectin, pectic acid, alginic acid, alginates, polyaminoacids, polyalcohols, polyglycols; and the like; and mixtures thereof. Any other coating material known to those of ordinary skill in the art is contemplated for inclusion in the coatings of the microcapsules set forth herein. In an exemplary embodiment, the coating material comprises alginic acid or an alginate.

The core may include one or more additional components other than the recombinant microbial cells. For example, the core may include a DOPA decarboxylase inhibitor. In certain embodiments, the decarboxylase enzyme inhibitor is carbidopa, a carbidopa prodrug, benserazide, methylphenidate, or a combination thereof.

### Formulations and Methods of Use

In one embodiment, a method for providing a subject with a treatment for Parkinson's disease is provided. In another embodiment, methods of treating depression or anxiety and methods of improving motivation to do difficult tasks are provided. The method comprises administering to the subject in need thereof an effective amount of a composition comprising a recombinant microbial cell of the invention. The composition may be a microencapsulated composition and/or a lyophilized composition.

Methods of treating other disorders associated with dopamine are also contemplated. The most common disease characterized by a dopamine production deficiency is Parkinson's disease; however, invention may be readily adapted for the treatment of other diseases characterized by insufficiency of dopamine production. In one embodiment of the invention, a method of treating a disorder resulting from dopamine-related dysfunction is provided.

In one embodiment, the method of the invention is intended for treating, preventing, managing and/or delaying the progression of Parkinson's disease, restless leg syndrome, depression, stress, obesity, chronic posttraumatic stress disorder, anxiety disorders, obsessive-compulsive disorders, postpartum depression; schizophrenia, narcolepsy, manic, bipolar, and affective disorder; executive function disorders, such as attention deficit disorder (ADHD), learning and memory disorders, Tourette syndrome and autism; cocaine, amphetamine, alcohol dependency, and addictive behavior, such as pathological gambling. The diseases and conditions enumerated above are given by way of example and not by way of limitation.

Pharmaceutical compositions comprising a recombinant microbial cell can be formulated to be suitable for oral administration, for example as discrete dosage forms, such as, but not limited to, tablets (including without limitation scored or coated tablets), pills, caplets, capsules, chewable tablets, powder packets, cachets, troches, wafers, aerosol sprays, or liquids, such as but not limited to, syrups, elixirs, solutions or suspensions in an aqueous liquid, a non-aqueous liquid, an oil-in-water emulsion, or a water-in-oil emulsion. Such compositions contain a predetermined amount of the pharmaceutically acceptable salt of the disclosed compounds, and may be prepared by methods of pharmacy well known to those skilled in the art. See generally, Remington: The Science and Practice of Pharmacy, 21st Ed., Lippincott, Williams, and Wilkins, Philadelphia, Pa. (2005).

In addition to the oral dosing, noted above, the compositions of the present invention may be administered by any suitable route, in the form of a pharmaceutical composition adapted to such a route, and in a dose effective for the treatment intended. The compositions may, for example, be administered parenterally, e.g., intravascularly, intraperitoneally, subcutaneously, or intramuscularly. For parenteral administration, saline solution, dextrose solution, or water may be used as a suitable carrier. In an embodiment of the invention, the therapeutic composition containing the recombinant microbial cells may be administered intrarectally. A rectal administration preferably takes place in the form of a suppository, enema, or foam.

In certain embodiments, the compositions comprising a recombinant microbial cell may be lyophilized. Lyophilization is an effective and convenient technique for preparing stable compositions that allow delivery of the cells. In some cases, the lyophilized composition is reconstituted prior to administration. In some cases, the reconstitution is by use of a diluent. Generally, at least one cryoprotectant is added to effectively lyophilize the composition. The cryoprotectants include, but are not limited to, mannitol, glycerol, dextrose, sucrose, and/or trehalose.

The compositions and methods described herein can be administered to a subject in need of treatment, e.g. in need of treatment for Parkinson's disease, depression, or anxiety. In some embodiments, the methods described herein comprise administering an effective amount of compositions described herein, e.g. recombinant microbial cells to a subject in order to alleviate a symptom. As used herein, “alleviating a symptom” is ameliorating any condition or symptom associated with a given condition. As compared with an equivalent untreated control, such reduction is by at least 5%, 10%, 20%, 40%, 50%, 60%, 80%, 90%, 95%, 99% or more as measured by any standard technique.

In certain embodiments, an effective dose of a composition comprising recombinant microbial cells as described herein can be administered to a patient once. In certain embodiments, an effective dose of a composition comprising recombinant microbial cells can be administered to a patient repeatedly. In some embodiments, the dose can be a daily administration, for example oral administration, of, e.g., a capsule comprising cells as described herein.

In some embodiments, the effective amount of the recombinant microbial cell is from about 10⁶ CFU to about 10¹³ CFU. Therefore, in some embodiments, the effective amount of the recombinant microbial cell is about 10⁶, about 10⁷, about 10⁸, about 10⁹, about 10¹⁰, about 10¹¹, about 10¹², or about 10¹³ CFU. In some preferred embodiments, the effective amount is about 10⁹ CFU of the recombinant microbial cell. In some embodiments, the effective amount results peak plasma levels similar to that of the standard tablet form of L-DOPA treatment. In some embodiments, the effective amount achieves stable therapeutic plasma L-DOPA concentrations of from about 300 to about 1600 ng/ml over time with the recombinant microbial cell as compared to traditional L-DOPA. Therefore, in some embodiments, the effective amount effective amount achieves stable therapeutic plasma L-DOPA concentrations of about 300 ng/ml, about 400 ng/ml, about 500 ng/ml, about 600 ng/ml, about 700 ng/ml, about 800 ng/ml, about 900 ng/ml, about 1000 ng/ml, about 1100 ng/ml, about 1200 ng/ml, about 1300 ng/ml, about 1400 ng/ml, about 1500 ng/ml, about 1600 ng/ml, or more. In some preferred embodiments, the effective amount results in peak plasma levels reaching about 1500 ng/ml. The optimal dose of the recombinant microbial cell maximizes gut colonization without inducing toxicity, including gut tissue damage, inflammation or gut microbial dysbiosis.

A composition comprising recombinant microbial cells can be administered over a period of time, such as over a 5 minute, 10 minute, 15 minute, 20 minute, or 25 minute period. The administration can be repeated, for example, on a regular basis, such as hourly for 3 hours, 6 hours, 12 hours, daily (i.e. one a day), every other day (i.e. on alternate days), or longer or such as once a week, or biweekly (i.e., every two weeks) for one month, two months, three months, four months or longer.

The dosage of a composition as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment, or make other alterations to the treatment regimen. The dosing, schedule can vary from once a week to daily depending on a number of clinical factors, such as the subject's sensitivity to recombinant microbial cells.

The desired dose or amount of activation can be administered at one time or divided into subdoses, e.g., 2-4 subdoses and administered over a period of time, e.g., at appropriate intervals through the day or other appropriate schedule. In some embodiments, administration can be chronic, e.g., one or more doses and/or treatments daily over a period of weeks or months. Examples of dosing and/or treatment schedules are administration daily, twice daily, three times daily or four or more times daily over a period of 1 week, 2 weeks, 3 weeks, 4 weeks, 1 month, 2 months, 3 months, 4 months, 5 months, or 6 months, or more.

The dosage ranges for the administration of recombinant microbial cells, according to the methods described herein depend upon, for example, the form of the cells, their potency, and the extent to which symptoms, markers, or indicators of a condition described herein are desired to be reduced, for example the percentage reduction desired. The dosage should not be so large as to cause adverse side effects. Generally, the dosage will vary with the age, condition, and sex of the patient and can be determined by one of skill in the art. The dosage can also be adjusted by the individual physician in the event of any complication.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage. A composition may be formulated such that a unit dose of the composition contains a specified number of microorganisms.

The efficacy of recombinant microbial cells in, e.g. the treatment of a condition described herein can be determined by the skilled clinician. However, a treatment is considered “effective treatment,” as the term is used herein, if any one or all of the signs or symptoms of a condition described herein are altered in a beneficial manner, other clinically accepted symptoms are improved, or even ameliorated, or a desired response is induced following treatment according to the methods described herein. Efficacy can be assessed, for example, by measuring a marker, indicator, symptom, and/or the incidence of a condition treated according to the methods described herein or any other measurable parameter appropriate. Efficacy can also be measured by a failure of an individual to worsen as assessed by hospitalization, or need for medical interventions (i.e., progression of the disease is halted). Methods of measuring these indicators are known to those of skill in the art and/or are described herein. Treatment includes any treatment of a disease in an individual or an animal (some non-limiting examples include a human or an animal) and includes: (1) inhibiting the disease, e.g., preventing a worsening of symptoms; or (2) relieving the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response. It is well within the ability of one skilled in the art to monitor efficacy of administration and/or treatment by measuring any one of such parameters, or any combination of parameters. Efficacy can be assessed in animal models of a condition described herein. When using an experimental animal model, efficacy of treatment is evidenced when a statistically significant change in a marker is observed.

The methods described herein can further comprise administering a second agent and/or treatment to the subject, e.g. as part of a combinatorial therapy. In certain embodiments of the present invention, the recombinant microbial cells can be used in combination therapy with at least one other therapeutic agent.

In certain embodiments, the pharmaceutical compositions, and methods for the treatment further comprise one or more therapeutic agents for treating Parkinson's disease selected from a dopaminergic agent, such as Levodopa-carbidopa (SINEMET®, SINEMET CR®) or Levodopa-benserazide (PROLOPA®, MADOPAR®, MADOPAR HBS®); a dopaminergic and anti-cholinergic agent, such as amantadine (SYMMETRYL®, SYMADINE®); an anti-cholinergic agent, such as trihexyphenidyl (ARTANE®), benztropine (COGENTIN®), ethoproprazine (PARSITAN®), or procyclidine (KEMADRIN®); a dopamine agonist, such as apomorphine, bromocriptine (PARLODEL®), cabergoline (DOSTINEX®), lisuride (DOPERGINE®), pergolide (PERMAX®), pramipexole (MIRAPEX®), or ropinirole (REQUIP®); a MAO-B (monoamine oxidase B) inhibitor, such as selegiline or deprenyl (ATAPRYL®, CARBEX®, ELDEPRYL®); a COMT (catechol O-methyltransferase) inhibitor, such as CGP-28014, tolcapone (TASMAR®) or entacapone (COMTAN®); or other therapeutic agents, such as baclofen (LIORESAL®), domperidone (MOTILIUM®), fludrocortisone (FLORINEF®), midodrine (AMATINE®), oxybutynin (DITROPAN®), propranolol (INDERAL®, INDERAL-LA®), clonazepam (RIVOTRIL®), or yohimbine.

The other therapeutic agent can be an anti-depression agent. Useful anti-depression agents include, but are not limited to, amitriptyline, clomipramine, doxepine, imipramine, triripramine, amoxapine, desipramine, maprotiline, nortriptyline, protripyline, fluoxetine, fluvoxamine, paroxetine, setraline, venlafaxine, bupropion, nefazodone, trazodone, phenelzine, tranylcypromine and selegiline. The anti-depression agent may be a norepinephrine reuptake inhibitors (SNRI). SNRIs include, but are not limited to, duloxetine (CYMBALTA®), desvenlafaxine (PRISTIQ®), levomilnacipran (FETZIMA®), and venlafaxine (EFFEXOR XR®).

The other therapeutic agent can be an anxiolytic agent. Useful anxiolytic agents include, but are not limited to, benzodiazepines, such as alprazolam, chlordiazepoxide, clonazepam, clorazepate, diazepam, halazepam, lorazepam, oxazepam, and prazepam; non-benzodiazepine agents, such as buspirone; and tranquilizers, such as barbituates.

Levodopa (L-DOPA), an aromatic amino acid, is a white, crystalline compound, slightly soluble in water, with a molecular weight of 197.2. It is designated chemically as (−)-L-a-amino-b-(3,4-dihydroxybenzene)propanoic acid. Its empirical formula is C₉H₁₁NO₄, and its structural formula is

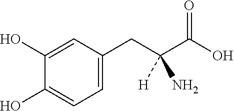 [SMILES: N[C@@H](Cc1ccc(O)c(O)c1)C(=O)O]

Current evidence indicates that symptoms of Parkinson's disease are related to depletion of dopamine in the corpus striatum. Administration of dopamine is ineffective in the treatment of Parkinson's disease apparently because it does not cross the blood-brain barrier. L-DOPA is able to cross the protective blood-brain barrier and enter the brain, where it is further converted into dopamine by the enzyme DOPA decarboxylase (DDC). Because L-DOPA can be converted into dopamine within the peripheral nervous systems, which may contribute to L-DOPA-related adverse side effects, L-DOPA is conventionally given in combination with a peripheral DDC inhibitor, such as carbidopa or benserazide to prevent its breakdown in the bloodstream, so more L-DOPA can enter the brain.

In some embodiments, the methods of the invention comprise co-administering a DOPA decarboxylase inhibitor. In certain embodiments, decarboxylase enzyme inhibitor is carbidopa, a carbidopa prodrug, benserazide, methylphenidate, or a combination thereof.

### EMBODIMENTS

The following embodiments also form part of the present disclosure:

1. A microencapsulated composition comprising a core component comprising a recombinant microbial cell capable of producing L-DOPA and colonizing the gut of a subject; and a coating material surrounding the core component.

2. The microencapsulated composition of embodiment 1, wherein the recombinant microbial cell is a probiotic.

3. The microencapsulated composition of embodiment 2, wherein the probiotic is E. coli Nissle 1917.

4. The microencapsulated composition of any one of embodiments 1-3, wherein the recombinant microbial cell comprises a heterologous hpaB and hpaC nucleotide sequence.

5. The microencapsulated composition of embodiment 4, wherein the hpaB and hpaC nucleotide sequence comprises SEQ ID NO: 1.

6. The microencapsulated composition of embodiment 4 or embodiment 5, wherein the hpaB and hpaC nucleotide sequence is stably integrated into the genome of the recombinant microbial cell.

7. The microencapsulated composition of embodiment 4 or embodiment 5, wherein the hpaB and hpaC nucleotide sequence is contained in a plasmid of the recombinant microbial cell.

8. The microencapsulated composition of any one of embodiments 4-7, wherein the hpaB and hpaC nucleotide sequence is operably linked to promoter sequence.

9. The microencapsulated composition of embodiment 8, wherein the promoter is a constitutive promoter or an inducible promoter.

10. The microencapsulated composition of embodiment 9, wherein the promoter is a rhamnose inducible promoter.

11. The microencapsulated composition of any one of embodiments 1-10, wherein the core component further comprises an aromatic amino acid- or DOPA-decarboxylase inhibitor.

12. The microencapsulated composition of embodiment 11, wherein the DOPA decarboxylase inhibitor is carbidopa or benserazide.

13. The microencapsulated composition of any one of embodiments 1-12, wherein the coating material comprises a polymer.

14. The microencapsulated composition of embodiment 13, wherein the coating material comprises alginic acid or an alginate.

15. The microencapsulated composition of any one of embodiments 1-14, further comprising a pharmaceutically acceptable carrier.

16. The microencapsulated composition of any one of embodiments 1-15, wherein the composition is lyophilized.

17. A recombinant microbial cell comprising a heterologous hpaB and hpaC nucleotide sequence stably integrated into the genome of the cell.

18. The recombinant microbial cell of embodiment 17, wherein the recombinant microbial cell produces L-DOPA and is capable of colonizing the gut of a subject.

19. The recombinant microbial cell of embodiment 17 or embodiment 18, wherein the recombinant microbial cell is a probiotic.

20. The recombinant microbial cell of embodiment 19, wherein said probiotic is E. coli Nissle 1917.

21. The recombinant microbial cell of any one of embodiments 17-20, wherein the hpaB and hpaC nucleotide sequence comprises SEQ ID NO: 1.

22. The recombinant microbial cell of any one of embodiments 17-21, wherein the hpaB and hpaC nucleotide sequence is operably linked to a promoter sequence.

23. The recombinant microbial cell of embodiment 22, wherein the promoter is a constitutive promoter or an inducible promoter.

24. The recombinant microbial cell of embodiment 23, wherein the promoter is a rhamnose inducible promoter.

25. A lyophilized composition comprising the recombinant microbial cell of any one of embodiments 17-24 and a cryoprotectant.

26. A microencapsulated composition comprising the recombinant microbial cell of any one of embodiments 39-46 and a coating material.

27. The microencapsulated composition of embodiments 26, wherein the coating material comprises a polymer.

28. The microencapsulated composition of embodiment 27, wherein the coating material comprises alginic acid or an alginate.

29. A method for treating Parkinson's disease comprising: administering to a subject in need thereof an effective amount of the microencapsulated composition of any one of embodiments 1-16 or 26-28, the recombinant microbial cell of any one of embodiments 17-24, or the lyophilized composition of embodiment 25.

30. A method of treating depression and/or anxiety or improving motivational performance comprising: administering to a subject in need thereof an effective amount of the microencapsulated composition of any one of embodiments 1-16 or 26-28, the recombinant microbial cell of any one of embodiments 17-24, or the lyophilized composition of embodiment 25.

31. The method of any one of embodiment 30, wherein the depression and/or anxiety is associated with Parkinson's disease, Parkinsonism, Alzheimer's disease, or other memory disorder.

32. A method of treating mild cognitive impairment or improving memory and learning comprising: administering to a subject in need thereof an effective amount of the microencapsulated composition of any one of embodiments 1-16 or 26-28, the recombinant microbial cell of any one of embodiments 17-24, or the lyophilized composition of embodiment 25.

33. The method of embodiment 32, wherein the mild cognitive impairment is associated with Parkinson's disease or Parkinsonism.

34. The method of embodiment 32, wherein the mild cognitive impairment is associated with Alzheimer's disease or other memory disorder.

35. The method of any one of embodiments 29-34, wherein said composition is administered orally.

36. The method of any one of embodiments 29-35, wherein the composition is administered twice daily, daily, or on alternate days.

37. The method of any one of embodiments 29-36, wherein the subject is a mammal.

38. The method of any one of embodiments 29-37, wherein the mammal is a human.

39. A method for ex vivo induction of L-DOPA production, the method comprising: providing a recombinant microbial cell comprising a heterologous hpaB and hpaC nucleotide sequence, wherein the hpaB and hpaC nucleotide sequence is operably linked to an inducible promoter; and incubating the recombinant microbial cell with an inducer of the promotor.

40. The method of claim 39, wherein the inducible promoter is a rhamnose inducible promoter and the inducer is rhamnose.

41. The method of claim 39 or claim 40, further comprising: administering to a subject in need thereof an effective amount of the induced recombinant microbial cell.

42. The method of claim 41, wherein the subject is suffering from Parkinson's disease, Parkinsonism, Alzheimer's disease, a memory disorder, depression, or anxiety.

43. The method of claim 41 or claim 42, wherein induced recombinant microbial cell is administered orally.

44. The method of any one of embodiments 41-43, wherein induced recombinant microbial cell is administered twice daily, daily, or on alternate days.

45. The method of any one of embodiments 41-44, wherein the subject is a mammal.

46. The method of any one of embodiments 39-45, wherein the mammal is a human.

All publications, patents and patent applications identified herein are incorporated by reference, as though set forth herein in full. The invention being thus described, it will be apparent to those skilled in the art that the same may be varied in many ways without departing from the spirit and scope of the invention. Such variations are included within the scope of the following claims.

The following examples are offered by way of illustration and not by way of limitation.

### EXAMPLES

### Example 1: Chromosome integrated HpaBC bacterial strains

To create an E. coli Nissle (EcN) strain to produce elevated levels of L-DOPA we first assembled recombinant plasmids to express a synthetic hpaBC gene construct. The hpaBC genes collectively encode 4-hydroxyphenylacetate-3-hydrolase, which converts L-Tyrosine to L-DOPA and are found naturally in selected E. coli strains. A codon-optimized variant was synthesized (Integrated DNA Technologies, Coralville IA). This new hpaBC variant shared 79% nucleotide sequence identity with the original genes and included additional bases on the 5′ and 3′ ends to facilitate cloning (SEQ ID NO: 1).

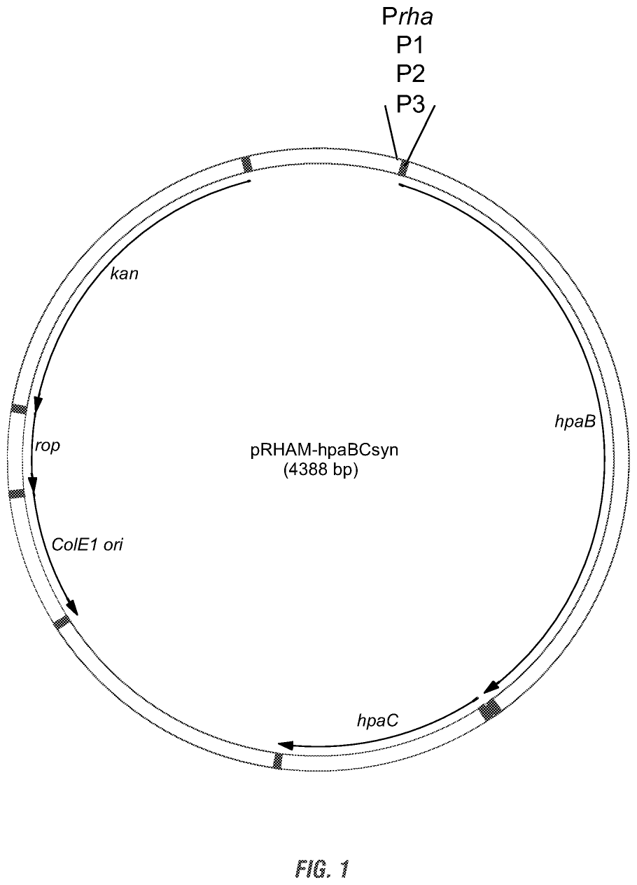

The synthetic hpaBC genes were cloned under control of the rhamnose (rha) promoter and operator for expression in EcN. For this, the commercially available pRHAM-C vector (Lucigen, Madison WI) was used. Kanamycin resistant transformants were screened for synthesis of L-Dopa in the presence of 1% rhamnose for dark colonies, indicative of oxidation of L-DOPA to dopachrome with subsequent polymerization to form the pigment melanin (Claus, H., and H. Decker. 2006. Bacterial tyrosinases. Syst. Appl. Microbiol. 29:3-14). Correct insertion of hpaBC into the pRham vector (vector pRham-hpaBC_{syn}; FIG. 1) was confirmed by characterizing plasmid DNA by restriction enzyme digestion and DNA sequencing.

The pRham-hpaBC_{syn} plasmid was introduced to EcN by chemical transformation. Additional variants of pRham-hpaBC_{syn} were also made to replace the rha promoter with promoters that allow constitutive expression of hpaBC. Three synthetic σ⁷⁰ promoter sequences (“parts” BBa_J23100 [P1], BBa_J23105 [P2], and BBa_J23111 [P3]) from the Anderson Promoter Collection (iGEM.org) were selected based on different levels of transcriptional activity afforded by each sequence. Each promoter sequences were incorporated into PCR primers used for inverse PCR reactions using pRham-hpaBC_{syn} as template DNA. Transformants constitutively expressing rhaBC were identified by colonies that turned dark brown following incubation in the absence of L-rhamnose. As predicted, expression of hpaBC from promoters P1, P2, and P3 resulted in different levels of constitutive L-DOPA production.

The plasmid-based EcN_{L-DOPA} is also described in U.S. Patent Application Pub. No. 20190262298, herein incorporated by reference.

hpaBC_{syn} nucleotide sequence (SEQ ID NO: 1):
GAAGGAGATATACATATGAAACCCGAAGATTTCCGTGCTTCAACACAGC
GCCCTTTCACTGGGGAAGAATACctgAAGAGCCTGCAAGACGGTCGTGA
AATTTATATTTACGGGGAGCGTGTGAAGGATGTTACGACCCATCCAGCC
TTTCGCAACGCCGCTGCGTCTGTGGCGCAGTTGTATGATGCGTTACACA
AACCTGAGATGCAGGATTCGTTGTGCTGGAACACAGACACGGGTTCGGG
AGGATATACTCATAAATTTTTTCGCGTTGCTAAGTCGGCAGACGACCTg
CGCCAACAACGTGATGCTATTGCTGAGTGGTCACGTCTGTCGTACGGGT
GGATGGGACGTACACCCGATTATAAAGCGGCGTTTGGATGCGCATTGGG
AGCTAACCCTGGATTCTATGGACAGTTCGAGCAGAATGCCCGCAACTGG
TACACACGCATTCAAGAAACTGGGTTGTATTTTAATCACGCCATTGTCA
ATCCGCCGATCGATCGCCACCTGCCCACGGATAAAGTAAAAGATGTATA
TATTAAGTTGGAAAAAGAGACAGACGCAGGGATCATTGTATCAGGCGCC
AAGGTGGTTGCGACCAATTCTGCCCTGACGCACTACAACATGATCGGCT
TTGGATCTGCTCAAGTGATGGGTGAAAACCCCGATTTTGCACTTATGTT
TGTAGCCCCCATGGACGCTGACGGGGTTAAACTGATTAGCCGCGCATCG
TACGAAATGGTCGCCGGGGCCACAGGCAGTCCGTACGATTATCCTTTAT
CTAGTCGCTTCGACGAAAACGACGCGATCTTAGTGATGGATAACGTCCT
GATTCCTTGGGAGAACGTCCTGATCTATCGTGATTTCGACCGCTGCCGT
CGTTGGACTATGGAAGGAGGCTTCGCTCGCATGTACCCTTTGCAAGCCT
GTGTACGCCTTGCTGTCAAACTTGATTTCATCACTGCGCTTTTGAAGAA
ATCGTTAGAGTGTACTGGGACGCTGGAGTTCCGTGGTGTCCAAGCCGAC
CTTGGCGAGGTGGTGGCTTGGCGTAATACTTTCTGGGCATTATCCGACT
CCATGTGCTCGGAAGCAACCCCCTGGGTCAATGGGGCATACCTTCCCGA
TCACGCCGCTCTTCAAACCTATCGCGTACTTGCGCCTATGGCTTATGCT
AAGATTAAAAATATTATCGAACGTAATGTGACTTCCGGCTTAATTTACT
TGCCCTCCAGCGCGCGCGATCTGAATAATCCTCAAATCGACCAGTATTT
AGCGAAGTATGTTCGCGGGAGCAACGGGATGGATCATGTCCAGCGCATC
AAAATCCTTAAGTTAATGTGGGATGCCATTGGTTCAGAATTTGGCGGGC
GTCATGAACTTTATGAAATTAATTACTCTGGCTCGCAAGACGAGATCCG
TctgCAATGCTTGCGCCAGGCGCAATCCTCGGGTAATATGGATAAGATG
ATGGCTATGGTAGACCGCTGCCTGTCCGAGTACGACCAAAACGGATGGA
CGGTCCCCCATCTGCATAACAACGATGACATTAACATGCTGGATAAGct
gctgAAATAACGCAGCAGGAGGTTAAGATGCAGcTGGACGAACAACGCc
tgCGCTTTCGTGACGCAATGGCGAGTTTGAGTGCAGCCGTTAATATCAT
TACAACAGAAGGGGACGCAGGTCAATGTGGAATCACTGCAACGGCCGTG
TGCTCAGTTACAGACACTCCGCCTTCATTAATGGTATGCATCAATGCTA
ACTCGGCTATGAATCCTGTCTTCCAAGGCAATGGGAAATTATGTGTGAA
CGTCCTGAACCATGAGCAAGAATGATGGCACGCCACTTCGCAGGCATGA
CAGGTATGGCAATGGAGGAGCGTTTTAGCTTGTCTTGCTGGCAAAAGGG
ACCActgGCCCAACCAGTTTTGAAGGGGTCTCTTGCATCATTAGAAGGG
GAAATCCGCGATGTCCAGGCGATTGGTACACACCTgGTTTACCTTGTCG
AGATCAAGAACATCATTTTATCCGCAGAGGGCCACGGGCTGATTTACTT
TAAACGCCGCTTCCATCCGGTTATGCTGGAAATGGAAGCTGCAATTTAA
GTAAGGAAACATTTATGCGCCTGCATCATCACCACCATCAC

Stable Integration of hpaBC_{syn} the EcN Chromosome

We improved the plasmid-based EcN_{L-DOPA} (termed EcN_{LDOPA}²) by stably integrating the hpaBC gene into the EcN genome (termed EcN_{LDOPA}⁴) to avoid the antibiotic selection requirements and potential loss of recombinant plasmids expressing hpaBC in vivo. To better titrate and then regulate L-DOPA production, we re-constructed the L-DOPA-expressing system using a rhamnose (Rha)-inducible, tightly regulated and tunable promoter. Since it is a non-metabolizable inert sugar, Rha (6-deoxy-L-mannose) is used clinically and as an inducer for bacterial gene regulation studies for its selective control of hpaBC expression in the mammalian gut with minimal disruption of resident microbiota. We reconstructed recombinant EcN by placing the synthetic hpaBC construct under control of the rhaBAD promoter and operator. The Rha-inducible hpaBC expression cassette was then integrated into the EcN chromosome to stabilize maintenance of the construct and eliminate the need for antibiotic selection required for recombinant plasmids.

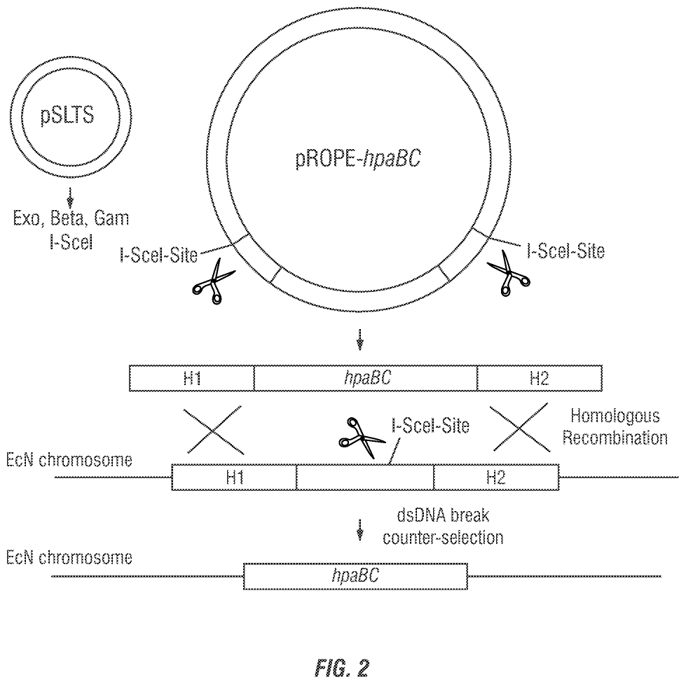

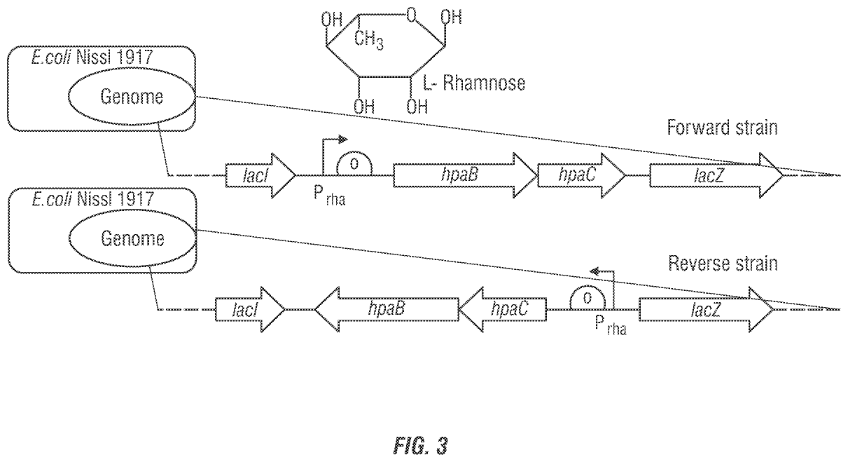

We developed a genetic system to stably integrate the genes into the chromosome of EcN, along with other bacterial strains. The ROPE (recombination of plasmid elements) genome editing system is a method that can efficiently integrate segments of DNA to a specific site within the E. coli chromosome using chromosome breakage as a counter-selection strategy (FIG. 2). This step is afforded by either expression of the I-SceI meganuclease (Kim, et al. 2014. BMC Biotechnology. 14:84) or by using CRISPR/Cas9.

To implement ROPE, we have engineered EcN such that the lac operator and promoter has been replaced by a DNA cassette containing a specific 15-bp sequence recognized by the I-SceI meganuclease, as described (Kim, et al. 2014. BMC Biotechnology. 14:84), and was accomplished by using λ Red recombination (KA Datsenko, BL Wanner. 2000. PNAS. v97:12, 6640-6645). We next cloned hpaBC, expressed by P1, P2, P3, and Pᵣₕₐ, into the “entry” vector pROPE by ligation independent cloning (C. J. Oster, G. J. Phillips. 2011. Plasmid. 66: 180-185). To facilitate this, the pROPE vector contains a cloning site flanked by regions of homology to the lac operon of EcN in a region that overlaps the promoter and operator. We selected the lac operon to integrate the hpaBC genes since it facilitates a convenient phenotypic screen for successful integration and its disruption is not known to reduce the fitness of EcN in the absence of lactose.

The resulting plasmid, pROPE-hpaBC_{syn} is transformed into the modified EcN strain with selection for kanamycin resistance. A second “helper” plasmid (pSLTS) is then transformed to EcN (ampicillin resistance) (Kim, et al. 2014. BMC Biotechnology. 14:84). The I-SceI meganuclease expressed from pSLTS cleaves the DNA specifically within the lac operator region. Expression of I-SceI is lethal to the EcN strain with the I-SceI meganuclease recognition site integrated into the chromosome unless the broken chromosome is repaired by homologous recombination with the linear DNA fragment that is liberated from pROPE, also by I-SceI cleavage (FIG. 2). Importantly, the modular nature of the expression and integration system allows engineering of EcN to express hpaBC, as well as other genes, at levels best suited for a specific biological or clinical outcome.

Since gene expression can be heavily influenced by the position of adjacent genes, we constructed two EcN strains by integrating hpaBC in both forward and reverse orientations with respect to the direction of the transcription of the lac operon (FIG. 3). We confirmed that the Rha-inducible hpaBC construct had correctly integrated into the targeted location (the lac operon) in both possible orientations on the E. coli chromosome by DNA sequencing. We also confirmed that our new EcN^{rha}_{L-DOPA} stably maintained and expressed the hpaBC genes over multiple passages.

### Dose-Dependent Rhamnose-Induced Production of L-DOPA Levels

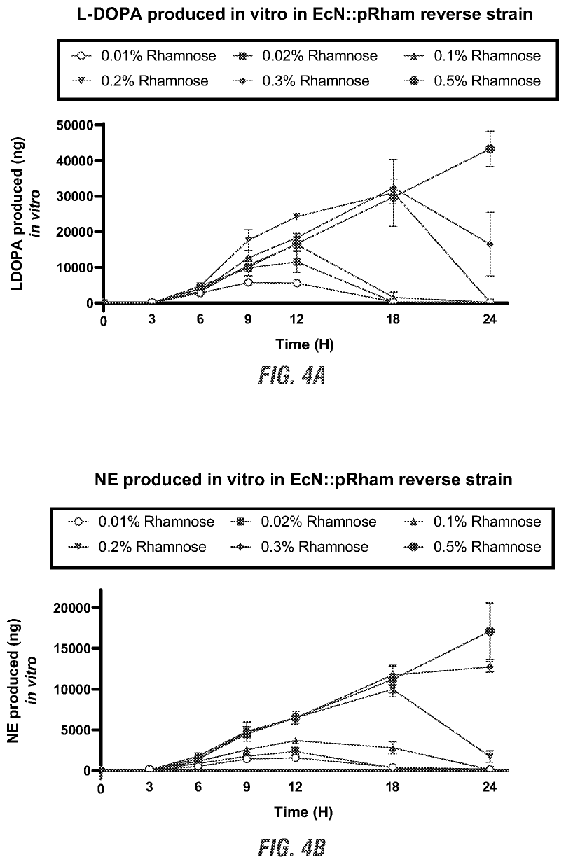

FIG. 4 shows the dose-dependent increase in total L-DOPA due to concentration effect of rhamnose inducer in EcN_{LDOPA}⁴ in vitro. EcN_{LDOPA} were streaked in LB agar plate and grown overnight. Single colony was obtained and grown in separate cultures with varied concentrations of rhamnose inducer (0.01, 0.02, 0.1, 0.2, 0.3, 0.5% (w/v)). Culture was harvested at 0, 3, 6, 9, 12, 18, 24 h and samples were processed for HPLC. Cumulative L-DOPA and norepinephrine (NE) were quantified and plotted.

Orally Administered EcN_{LDOPA}^{GEN 2/4} is Efficacious in Attenuating Spatial Learning and Memory Deficits in MitoPark Mice

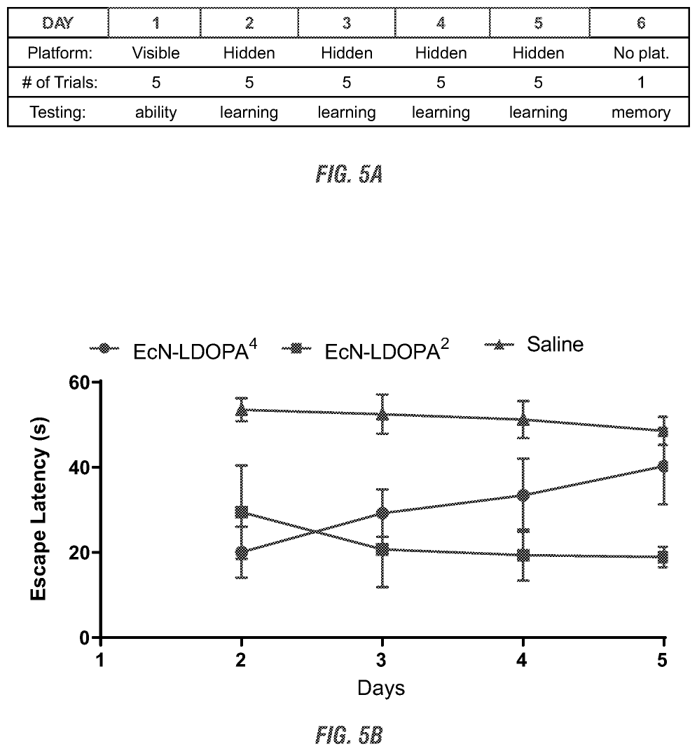

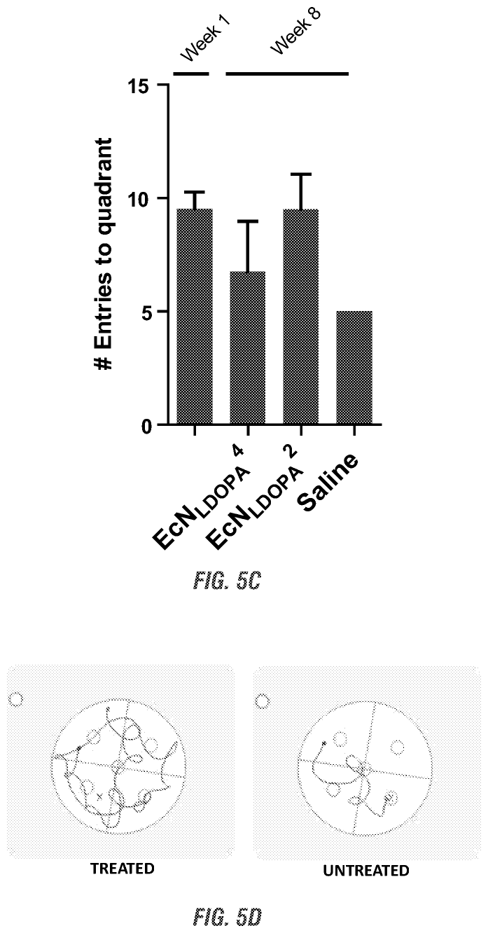

MitoPark (13-15 wk) mice were orally administered with EcN_{LDOPA}⁴, EcN_{LDOPA}² (respective doses of 1-2×10¹⁰ CFU) or saline every 12 h with benserazide (40 mg/kg) for 8 wk. Spatial learning and memory deficits were evaluated using the Morris water maze cognitive test and values obtained 8 wk post-treatment were plotted. Escape latency was measured as a function of spatial learning. Both EcN_{LDOPA}⁴ and EcN_{LDOPA}² (n=3,4) groups demonstrated moderate to high improvement in learning compared to saline-treated (n=8) animals. Memory retention between the EcN strains improved as well compared to saline-treated animals, further suggesting both EcN_{LDOPA}⁴ and EcN_{LDOPA}² improve neurocognitive deficits typically associated with MitoPark mice (FIG. 5).

Orally Administered EcN_{LDOPA}^{GEN 2/4} is Efficacious in Attenuating Locomotor Deficits in MitoPark Mice

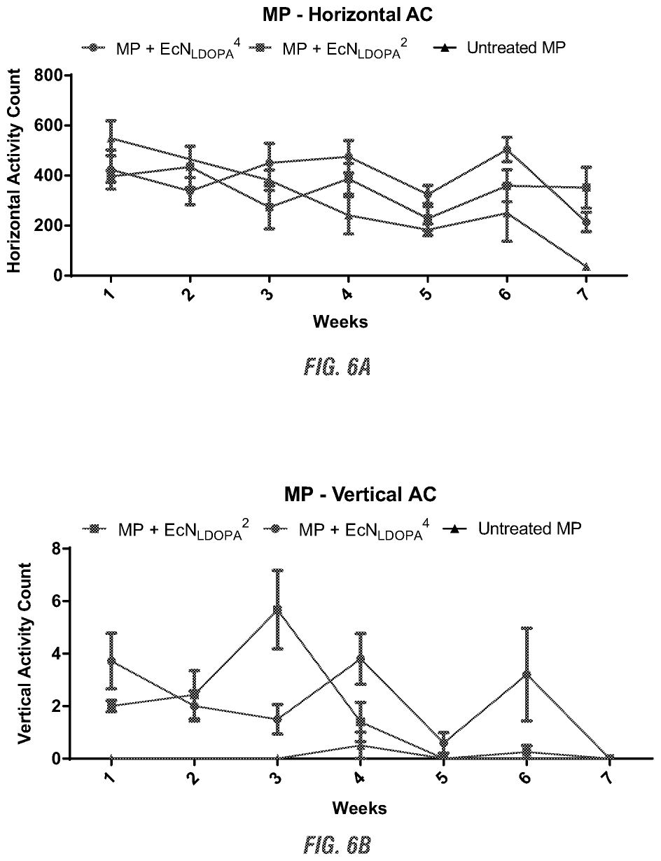

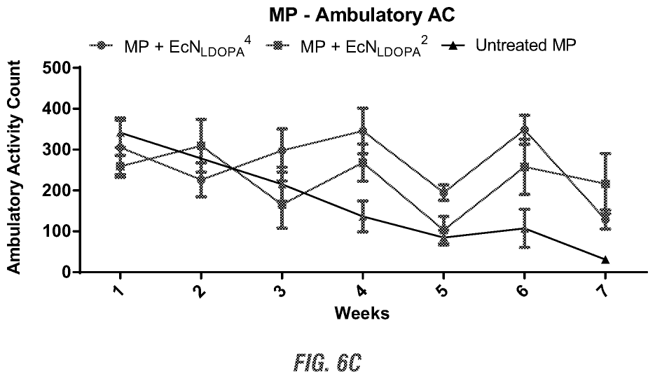

MitoPark (13-15 wk) mice were orally administered with EcN_{LDOPA}⁴ or EcN_{LDOPA}² (respective doses of 1-2×10¹⁰ CFU) or saline every 12 h with Benserazide (40 mg/kg) for 8 wk. Locomotor activity including total horizontal activity, vertical activity and ambulatory activity counts were measured using VersaMax and plotted (n=4-7). As shown in FIG. 6, horizontal, vertical and ambulatory activity plots illustrate overall improvement of total activity counts in MitoPark treated with EcN_{LDOPA}⁴ or EcN_{LDOPA}² as compared to saline-treated animals.

Collectively, these results demonstrate the precise and temporal control of L-DOPA production without the use of recombinant plasmids, which can be unstable even with continued selection by antibiotics. These data also suggest that sufficient L-DOPA can be produced from a single-copy integration system.

### Example 2: Ex Vivo Induction

We next tested the reverse EcN^{rha}_{L-DOPA} strain in a pilot study of C57BL/6NCrl mice to evaluate whether it produces therapeutic plasma levels of L-DOPA in vivo by adopting an ex vivo pre-induction step. This ex vivo Rha pre-activation procedure does not require oral administration of the inducer. Briefly, EcNᵣₕₐ^{L-DOPA} was grown overnight in LB medium until late exponential phase, followed by pre-induction with the addition of 0.5% Rha for 9 h. The pre-activated bacterial pellets were concentrated 20-fold in formulation buffer (2.28 g/L KH₂PO₄ and 14.5 g/L K₂HPO₄ in 15% glycerol, pH 7.5) and stored at −80° C. For administration, cells were thawed and resuspended in formulation buffer. We treated mice (3/group) with a single p.o. dose of 2×10¹⁰ CFU of pre-activated EcN^{rha}_{L-DOPA} in formulation buffer and Bz (40 mg/kg). Plasma L-DOPA levels were determined by HPLC from samples collected at baseline and 6 h post-treatment. A human therapeutic level of L-DOPA (770 ng/mL) was rapidly seen by 6 h post-EcN^{rha}_{L-DOPA}, suggesting EcN^{rha}_{L-DOPA} can also more efficiently produce L-DOPA in vivo than the plasmid-based system, in which a human therapeutic level of L-DOPA can only been seen 8-16 h post-treatment.

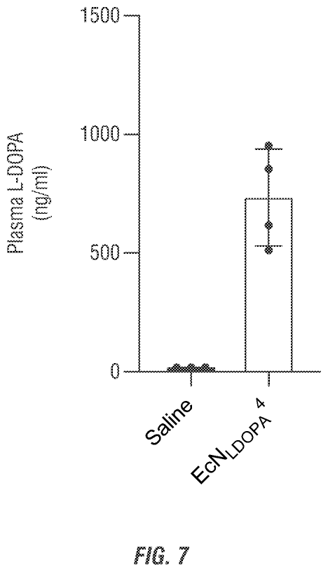

FIG. 7 shows elevated plasma L-DOPA post daily treatment of EcN_{LDOPA}⁴ in the C57BL/6 black mouse model. EcN_{LDOPA}⁴ pre-induced with Rhamnose was orally administered C57BL/6 mice daily for three weeks along with benserazide. Saline treated group was used as controls. Plasma L-DOPA measured by HPLC-ECD.

### Example 3: Lyophilization Strategy for Steady-State Delivery

We applied another formulation strategy by lyophilizing bacteria using 50% sucrose as cryoprotectant to ensure long-term stability of our EcN biotherapeutics and its endogenous ability to release L-DOPA post-lyophilization.

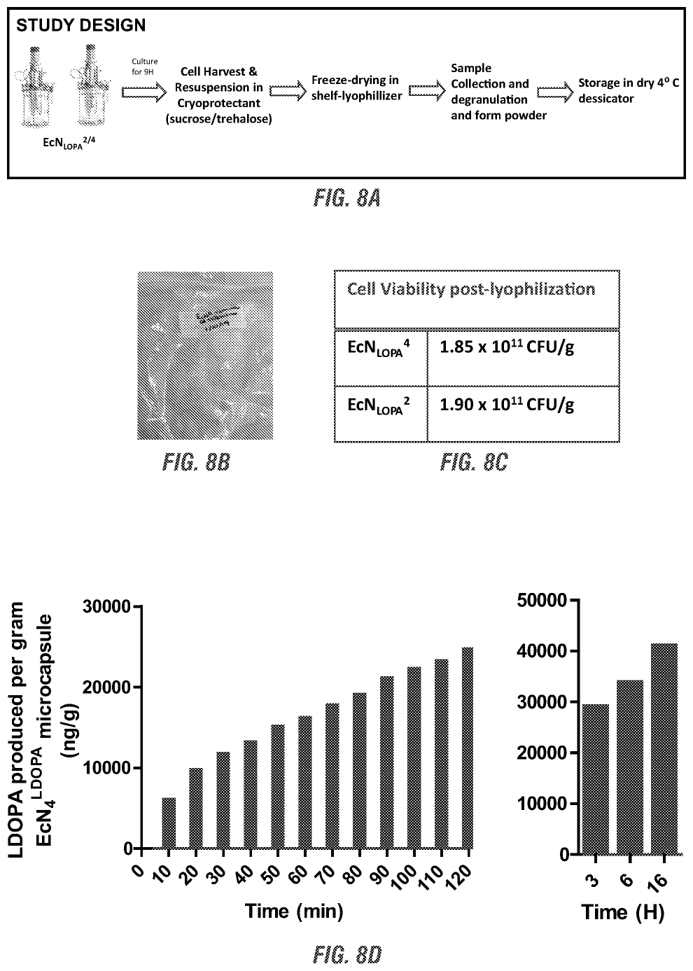

FIG. 8 shows EcN_{LDOPA}² and EcN_{LDOPA}⁴ viability and release kinetics of L-DOPA from lyophilized formulation. Both generations of EcN_{LDOPA} were grown overnight and harvested the following day. The cells were resuspended in 50% (w/v) of cryoprotectant such as sucrose/trehalose and was placed on stainless steel trays in −80° C. overnight. Subsequently, the pre-filled trays were placed in shelf-lyophilizer for up to 72 h to ensure efficient freeze-drying. The contents of the tray were then collected, degranulated by using rubber mallet and stored in 4° C. desiccator until use. Cell viability was measured using plate-counting method and data were normalized to the amount (g) of respective bacterial strains. Time-dependent production of L-DOPA from lyophilized bacteria was measured by HPLC and plotted. Data were normalized to per gram of respective bacterial strains.

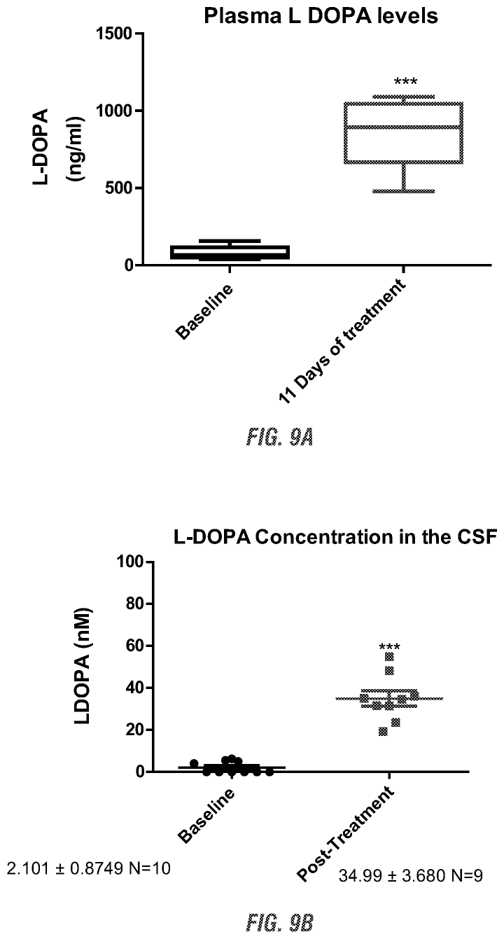

FIG. 9 shows administration of liquid formulated EcN⁴_{-LDOPA} increased L-Dopa level in dog plasma and CSF. Plasma L-Dopa level was determined in 10 healthy beagle dogs (5 males and 5 females) in baseline and at 11 days post administration of EcN⁴_{-LDOPA} and benserazide orally twice a day. Each group represent mean quantity (ng/ml) in plasma measured by HPLC-EC (N=10). Significant increase in plasma L-Dopa was observed in the treated group as compared basal line level from (N=10), t test p<0.0001 (FIG. 9A). Collected CSF samples at baseline and post-treatment of EcN⁴_{-LDOPA} was subjected for HPLC-ECD analysis. A Significant increase in L-Dopa level was noticed after treatment (p<0.0001, N=9) as compared to baseline (FIG. 9B).

### Example 4: Microencapsulation of EcN_{LDOPA}⁴ with Nanoparticle Using Calcium-Alginate for Continuous Steady-State Delivery

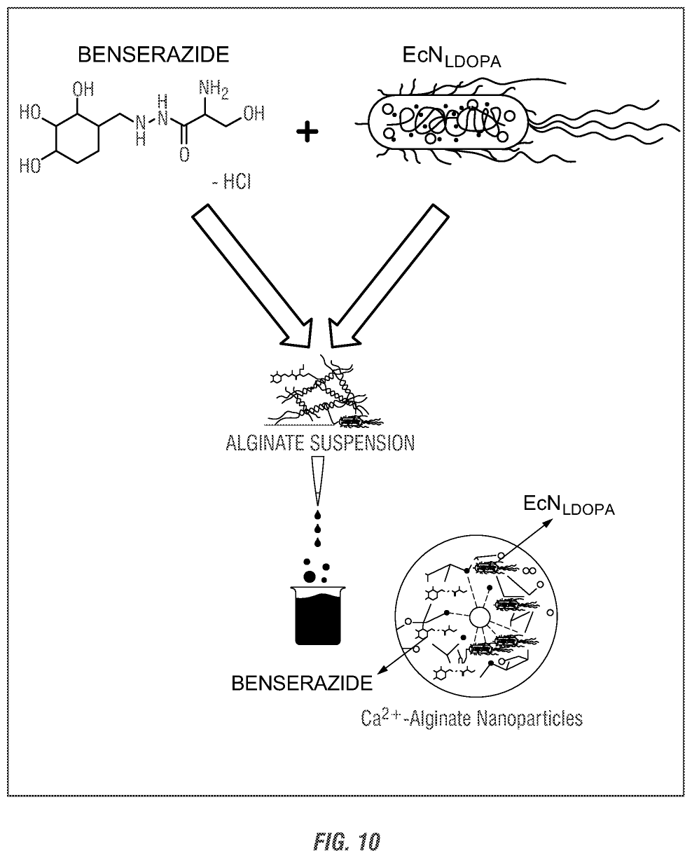

With current existing pharmacological intervention predominantly focused on small molecule delivery, we proposed a new strategy of drug delivery using EcN_{LDOPA} biotherapeutics in combination with/without Benserazide as a single microcapsule nanoparticle using calcium-alginate (FIG. 10). Microencapsulation of EcN with cross-linking nanopolymers can protect against low pH, a high bile-salt concentration and high temperatures. Additionally, encapsulation of EcN can serve as prebiotics for improved colonization and therapeutic efficacy.

We microencapsulated novel genetically engineered EcN_{LDOPA}⁴ with calcium alginate to a) improve survivability of a live biotherapeutics and b) direct, sustained release of L-DOPA and Benserazide. By harnessing the potential of a natural, cross-linking polymer such as alginate and utilizing it as a microcapsule for sustained and regulated release of a continuously producing EcN biotherapeutics and small molecule is a novel PD-based therapy.

Release kinetics of Escherichia coli Nissle 1917 (EcN) genetically engineered to produce L-DOPA (EcN LD op A) in calcium-alginate microcapsule are shown in FIG. 11. EcN_{LDOPA} was grown overnight, harvested and resuspended in 0.1% Peptone solution. Cell suspension was mixed with 2% (w/v) sodium alginate, stirred uniformly and injected in a dropwise manner in 0.1 M calcium chloride. Following 30 min of gelation, the calcium-alginate beads containing EcN_{LDOPA} were washed, collected and stored in 0.1% peptone in 4° C. For determining the release kinetics, EcN_{LDOPA} microcapsules were mixed with 1% sodium citrate dihydrate and the number of cumulative colony-forming units (CFUs) released were plotted against time. Linear regression analysis was performed to determine the rate of release of EcN from alginate microcapsules.

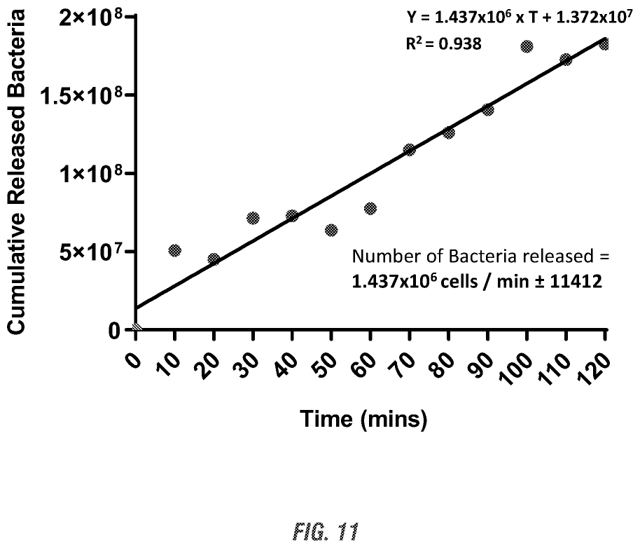

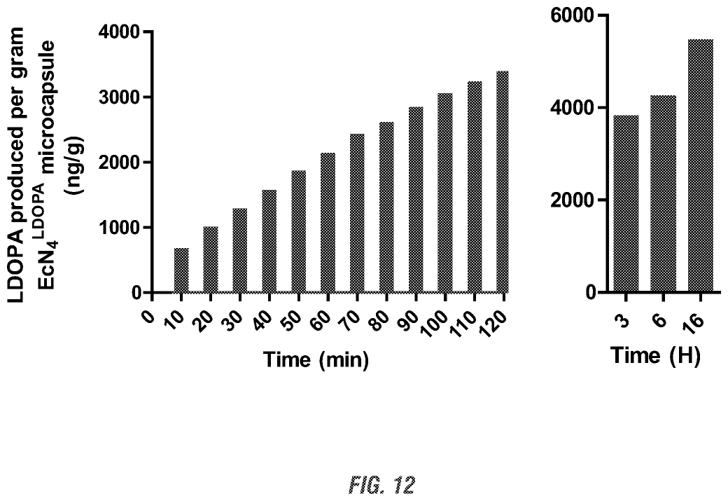

L-DOPA release kinetics from calcium-alginate EcN_{LDOPA} microcapsules are shown in FIG. 12. To measure the cumulative amount of L-DOPA released from EcN_{LDOPA} microcapsules, the microcapsules were dissolved in 1% sodium citrate dihydrate. A certain volume of suspension was collected every 10 min up to 2 h and 3, 6 and 16 hours. Samples were subjected to protein precipitation with an antioxidant solution followed by HPLC quantification. Released L-DOPA levels were normalized to weight of EcN_{LDOPA} microcapsules and plotted against respective time of collections.

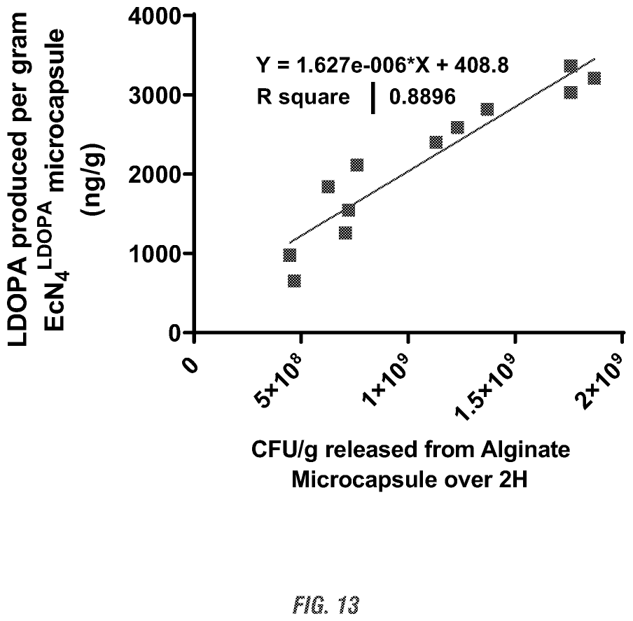

Release kinetics of L-DOPA from respective number of EcN_{LDOPA} calcium-alginate microcapsules are shown in FIG. 13. To measure the cumulative amount of L-DOPA released from EcN_{LDOPA} released from calcium-alginate microcapsules, the microcapsules were dissolved in 1% sodium citrate dihydrate. A certain volume of suspension was collected every 10 min for up to 2 h. Samples were subjected to protein precipitation with an antioxidant solution followed by HPLC quantification. Data for L-DOPA concentration was normalized to weight of EcN_{LDOPA} microcapsules and plotted against the number of CFU released per gram of EcN_{LDOPA} microcapsules.

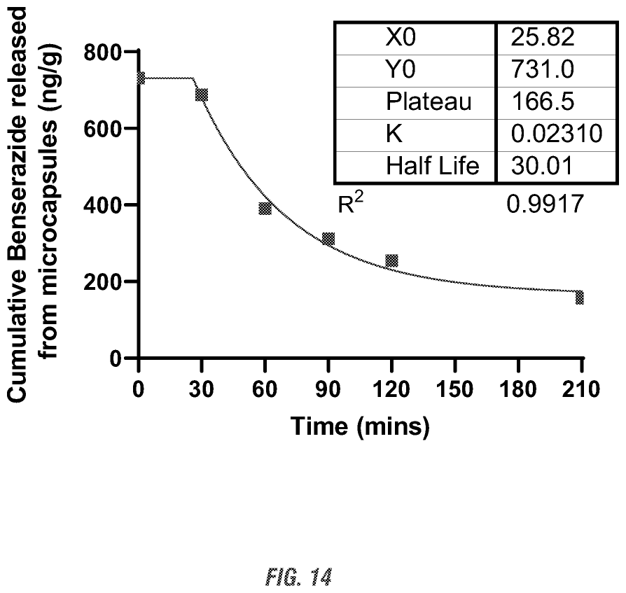

Release kinetics of benserazide from calcium-alginate microcapsules are shown in FIG. 14. 100 mM benserazide was weighed, dissolved in 0.1% Peptone solution, stirred uniformly with 2% (w/v) sodium alginate, and injected in a dropwise manner in 0.1 M calcium chloride. Following 30 min of gelation, the benserazide-alginate beads were washed, collected, and stored in 0.1% peptone in 4° C. For determining the release kinetics, a certain weight of benserazide microcapsules was added in 1% sodium citrate dihydrate and placed in shaker at 230 RPM. The amount of benserazide released in media was quantified by HPLC and plotted against respective collection times. Non-linear regression for plateau and one-phase decay analysis were performed to determine the rate of release and half-life of benserazide from alginate microcapsules.

### Example 5: Administration of EcN_{LDOPA} in Animal Model of Parkinson's Disease (PD)

### Generations of EcN_{LDOPA}

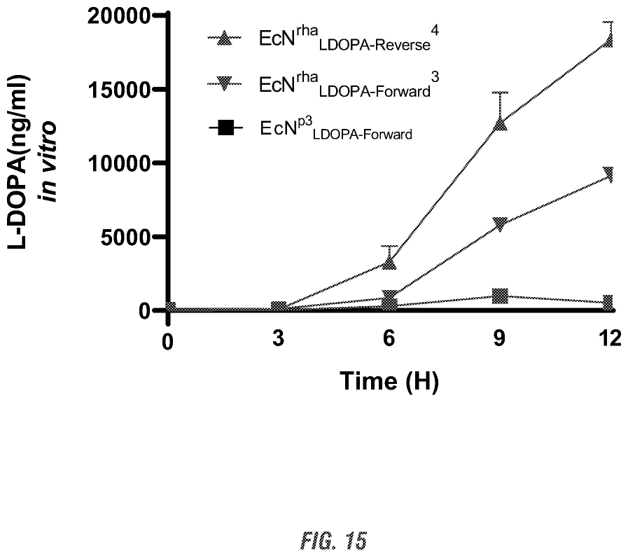

The generations of EcN_{LDOPA} engineered to develop lead therapeutic are shown in FIG. 15. EcN^{rha}_{LDOPA-Reverse}⁴ is the Rhamnose inducible EcN_{LDOPA} with hpaBC in reverse orientation. EcN^{rha}_{LDOPA-Forward}³ is the Rhamnose inducible EcN_{LDOPA} with hpaBC in forward orientation. EcN^{p3}_{LDOPA-Forward} is the constitutively expression EcN_{LDOPA} with synthetic p3 promoter and hpaBC in forward orientation.

### EcN_{LDOPA} Significantly Increases Striatal DA in C57BL/6 Following Single Administration.

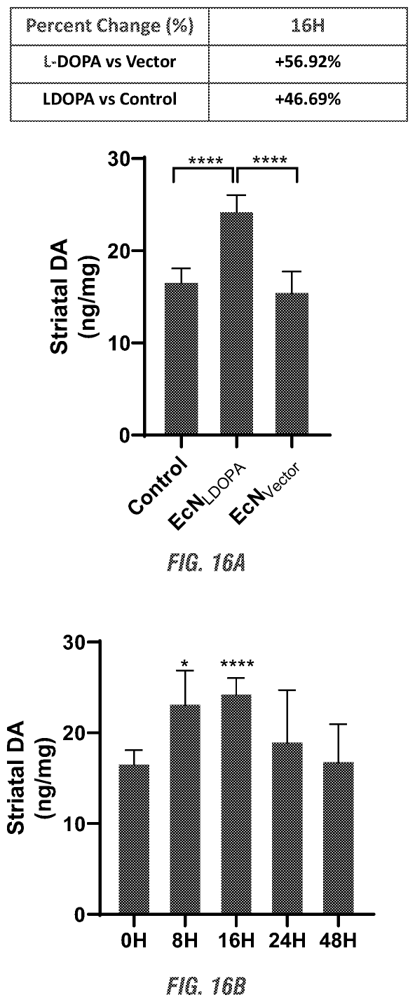

C57BL rodents were orally administered a single dose of pre-activated EcN_{LDOPA} with Benserazide (40 mg/kg). EcN_{LDOPA} administration significantly increased (p<0.0001) striatal DA in both untreated control (46.7%) and EcN_{vector} (56.9%) at 16H (FIG. 16A) with steady decrease to baseline at 24H and 48H (FIG. 16B).

### EcN_{LDOPA} Significantly Rescues Locomotor Deficits in MitoPark Animal Model of PD.

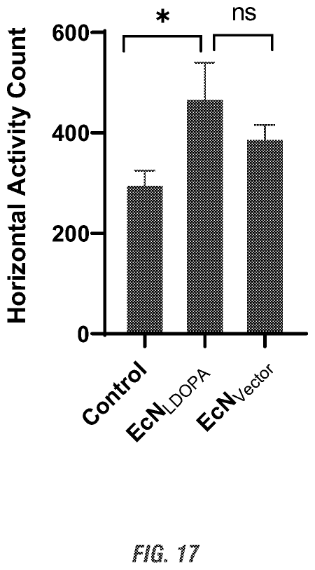

MitoPark (20-22 wk) mice were chronically administered with either pre-activated EcN_{LDOPA}⁴ or EcN_{vector} with Benserazide (40 mg/kg) for 10 Days. Locomotor activity was recorded using VersaMax. Horizontal activity counts were evaluated and plotted. EcN_{LDOPA} (n=11) significantly improves (*p<0.05) compared to untreated control Mitopark (n=38), with no significant change observed in EcN_{vector} (n=9) treated MitoPark (p=0.62) (FIG. 17).

### EcN_{LDOPA} Moderately Improves Depressive-Like Behavior in MitoPark Animal Model of PD.

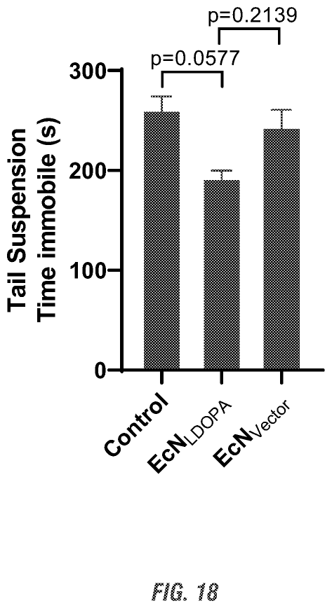

MitoPark (20-22 wk) mice were chronically administered with either pre-activated EcN_{LDOPA}⁴ or EcN_{Vector} with Benserazide (40 mg/kg) for 10 Days. Depressive-like behavior was evaluated between untreated Control (n=16), EcN_{LDOPA} (n=6) and EcN_{vector} (n=12) using Tail Suspension Test (FIG. 18). Animals were video recorded and behavioral output measure of time immobile was evaluated using AnyMaze v.6.3.

### Chronic Administration of EcN_{LDOPA} Ensures Stable Colonization Profile in MitoPark Animal Model of PD.

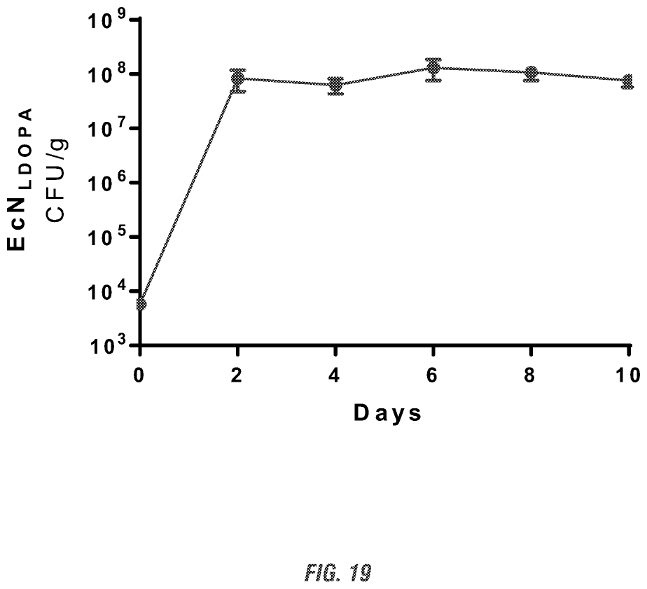

MitoPark (20-22 wk) mice were chronically administered with pre-activated EcN_{LDOPA}⁴ with Benserazide (40 mg/kg) for 10 Days. Fecal pellets were collected on Day 0, 2, 4, 6, 8 and 10 and quantified using targeted-qPCR. EcN_{LDOPA} was highly abundant and showed to have effectively colonized in the gut (FIG. 19).

### Plasma L-DOPA Chronic Dose Pharmacokinetic Profile in MitoPark Animal Model of PD.

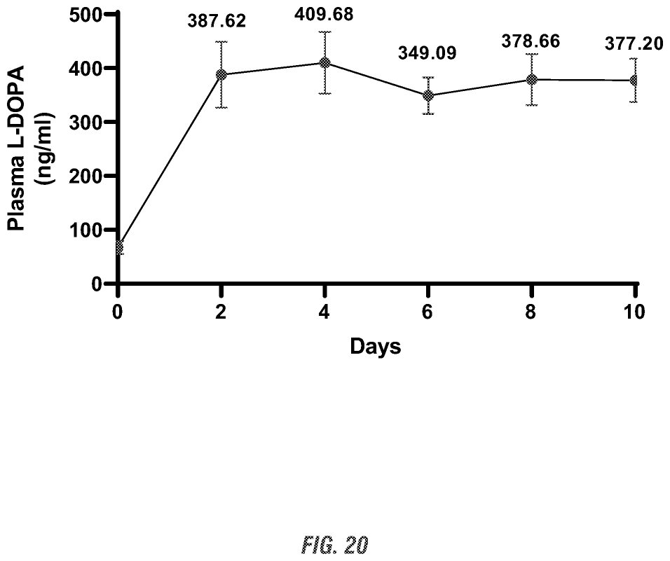

MitoPark (20-22 wk) mice were chronically administered with pre-activated EcN_{LDOPA}⁴ with Benserazide (40 mg/kg) for 10 Days. Plasma was collected at days (0, 2, 4, 6, 8, 10). Levodopa (ng/ml) was measured using HPLC-ECD, quantified using a standard curve and corrected with an internal standard correction factor (FIG. 20). Animals were age matched and both males and females are included (n=6/timepoint).

### EcN_{LDOPA} Significantly Improves Dopamine and Norepinephrine Neurochemical Profile in MitoPark Animal Model of PD Following Chronic Administration.

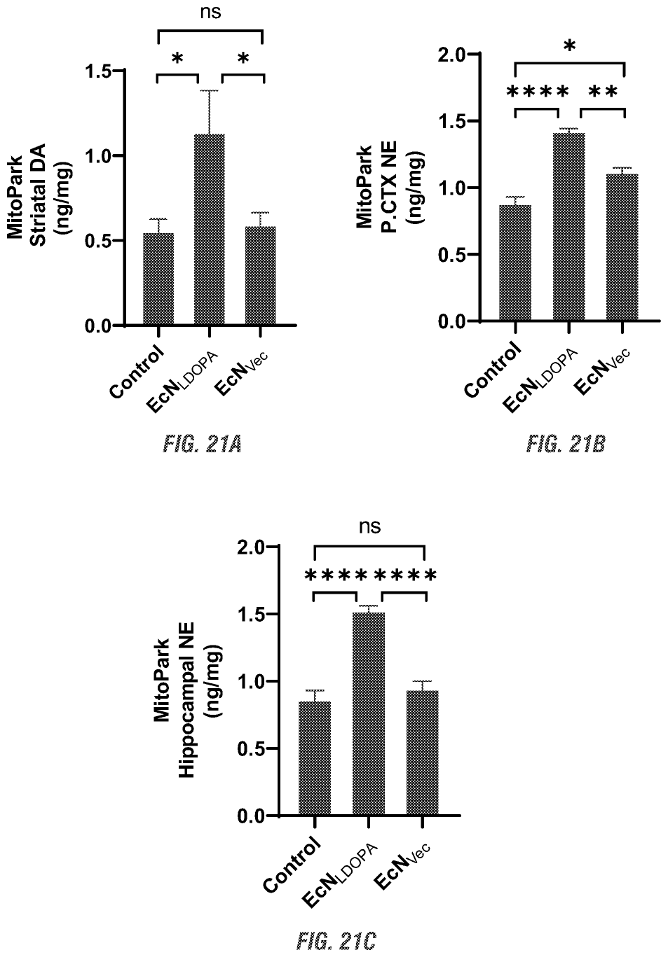

MitoPark (20-22 wk) mice were chronically administered with either pre-activated EcN_{LDOPA}⁴ or EcN_{Vector} with Benserazide (40 mg/kg) for 10 Days. Brain striatum (STR) (FIG. 21A), pre-frontal cortex (P.FCTX) (FIG. 21B) and hippocampus (FIG. 21C) were processed for HPLC-ECD and neurochemical DA and NE were quantified using a standard curve. All samples concentration were corrected using internal standard correction factor and normalized with wet tissue weight. EcN_{LDOPA} administration significantly increased striatal DA for both untreated control (p=0.017), and EcN_{Vector} (p=0.026). This effect was similarly observed in Prefrontal Cortex with EcN_{LDOPA} significantly elevated P.FCTX NE and Hippocampal NE compared to untreated control and EcN_{vector}.

### Example 6: APP-KI Rodent Model of Alzheimer's Disease (AD)

Significant Levels of EcN_{L-DOPA} were Detected in Fecal Samples of APP-KI Rodents.

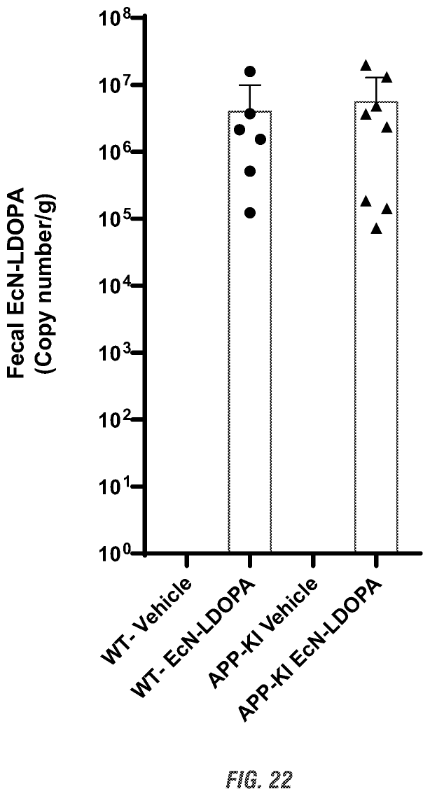

Lyophilized EcN_{LDOPA} pre-activated with Rhamnose was co-administered with Benserazide (40 mg/kg) to APP-KI rodent model and its wild-type littermate control for approximately two weeks. Fecal pellets were collected at the end of two weeks and quantified with targeted-qPCR. Copy number for synthetic-hpaB/C was quantified using a standard curve and normalized to animal's respective fecal weight. Quantified plots of WT and APP-KI animals treated with either EcN_{LDOPA} or vehicle (formulation buffer) are shown in FIG. 22.

### EcN_{L-DOPA} Improves Hippocampal Dopamine Levels in APP-KI Rodents.

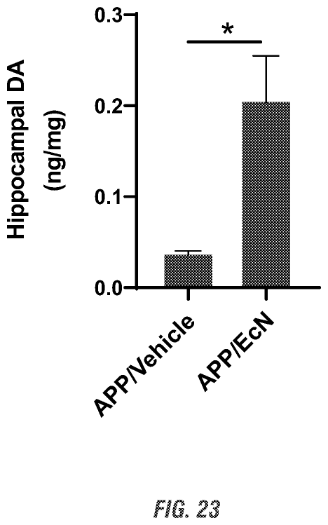

Lyophilized EcN_{LDOPA} pre-activated with Rhamnose was co-administered with Benserazide (40 mg/kg) to APP-KI rodent model and its wild-type littermate control for approximately two weeks. Brain hippocampus was processed for HPLC-ECD and neurochemicals were quantified using a standard curve. All samples concentration were corrected using internal standard correction factor and normalized with wet tissue weight. EcN_{LDOPA} significantly increases (p<0.05) hippocampal dopamine concentration, by approximately 5.8-fold the vehicle administered APP-KI rodents (FIG. 23).

### EcN_{L-DOPA} Improves Pre Frontal Cortex Norepinephrine Levels in APP-KI Rodents.

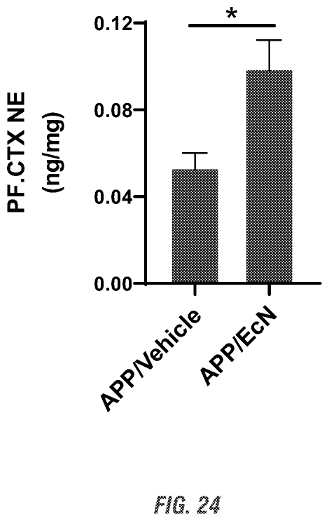

Lyophilized EcN_{LDOPA} pre-activated with Rhamnose was co-administered with Benserazide (40 mg/kg) to APP-KI rodent model and its wild-type littermate control for approximately two weeks. Brain pre-frontal cortex was processed for HPLC-ECD and neurochemicals were quantified using a standard curve. All samples concentration were corrected using internal standard correction factor and normalized with wet tissue weight. EcN_{LDOPA} significantly increases (p<0.05) pre-frontal cortex norepinephrine concentration, by approximately 1.56-fold the vehicle administered APP-KI samples (FIG. 24).

### EcN_{L-DOPA} Effectively Colonized in the Gut of LC-Lesioned Tg 344 AD Rats.

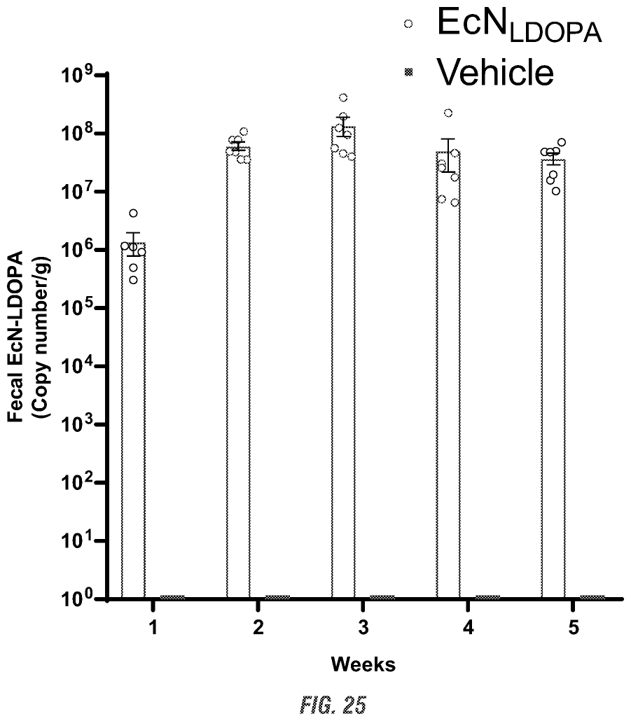

Locus Coeruleus-Lesioned Tg 344 AD rats were administered EcN_{LDOPA} with Benserazide (40 mg/kg) daily for 5 weeks. Fecal pellets were collected weekly and quantified using targeted-qPCR. Copy number for synthetic-hpaB/C was quantified using a standard curve and normalized to the animal's respective fecal weight (copy number/g of feces). EcN_{LDOPA} was highly abundant in feces, suggesting the strain effectively colonized in the rat gut (FIG. 25).

EcN_{L-DOPA} Increases Plasma L-DOPA in LC-Lesioned Tg344 Rat Model.

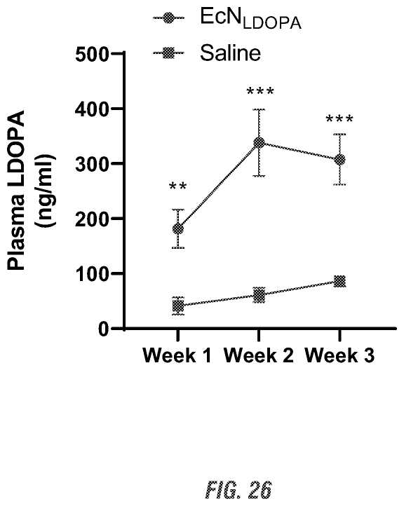

Locus Coeruleus-Lesioned Tg 344 AD rats were administered EcN_{LDOPA} with Benserazide (40 mg/kg) daily for 5 weeks. Blood was collected weekly and processed for HPLC-ECD. L-DOPA was quantified using a standard curve and corrected with an internal standard correction factor. Animals were age matched with both sexes included (n=6-7/timepoint) (FIG. 26).

### Ec_{NL-DOPA} Dramatically Increased Pre Frontal Cortical NE Levels in LC-Lesioned Tg344 Rat Model.

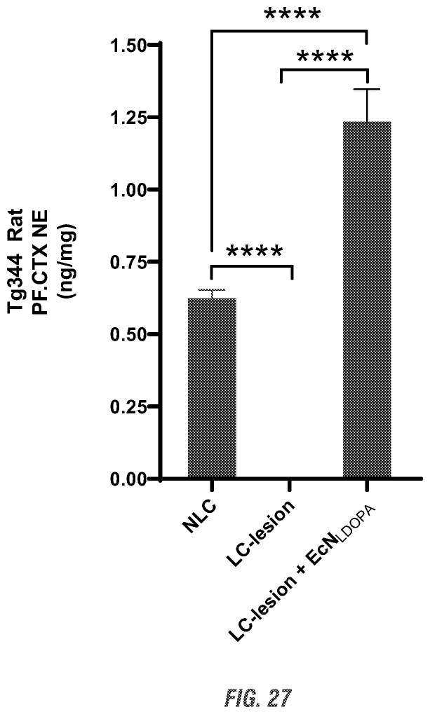

Locus Coeruleus-Lesioned Tg 344 AD rats were administered EcN_{LDOPA} with Benserazide (40 mg/kg) daily for 5 weeks. Pre-frontal cortical (P.FCTX) Norepinephrine (NE) was compared against non-lesioned LC (NLC) and Saline administered Lesioned Control (LC-lesion) (n=5-9). Prefrontal cortices were processed for HPLC-ECD and NE were quantified using a standard curve. All samples concentration were corrected using internal standard correction factor and normalized with wet tissue weight. EcN_{LDOPA} administration completely recovers prefrontal cortical NE in LC-lesioned Tg 344 rats compared to NLC and LC-lesion rats (FIG. 27).

1-48. (canceled)

49. A method for treating Parkinson's disease, depression and/or anxiety, or mild cognitive impairment or improving motivational performance or memory and learning, the method comprising:
administering to a subject in need thereof an effective amount of the recombinant microbial cell comprising a heterologous hpaB and hpaC nucleotide sequence stably integrated into the genome of the cell.

50. The method of claim 49, wherein said composition is administered orally.

51. The method of claim 49, wherein the composition is administered twice daily, daily, or on alternate days.

52. The method of claim 49, wherein the subject is a mammal.

53. The method of claim 52, wherein the mammal is a human.

54. The method of claim 49, wherein the depression and/or anxiety or the mild cognitive impairment is associated with Parkinson's disease, Parkinsonism, Alzheimer's disease, or other memory disorder.

55. The method of claim 49, wherein the recombinant microbial cell produces L-DOPA and is capable of colonizing the gut of a subject.

56. The method of claim 49, wherein the recombinant microbial cell is a probiotic.

57. The method of claim 56, wherein said probiotic is E. coli Nissle 1917.

58. The method of claim 49, wherein the hpaB and hpaC nucleotide sequence comprises SEQ ID NO: 1.

59. The method of claim 49, wherein the hpaB and hpaC nucleotide sequence is operably linked to a promoter sequence.

60. The method of claim 58, wherein the promoter is a constitutive promoter or an inducible promoter.

61. The method of claim 59, wherein the promoter is a rhamnose inducible promoter.

62. A method for treating Parkinson's disease, depression and/or anxiety, or mild cognitive impairment or improving motivational performance or memory and learning, the method comprising:
administering to a subject in need thereof an effective amount of a microencapsulated composition comprising;
a core component of a recombinant microbial cell comprising a heterologous hpaB and hpaC nucleotide sequence stably integrated into the genome of the cell; and
a coating material surrounding the core component.

63. The method of claim 61, wherein the core component further comprises a DOPA-decarboxylase inhibitor.

64. The method of claim 62, wherein the DOPA decarboxylase inhibitor is carbidopa or benserazide.

65. The method of claim 61, wherein the coating material comprises a polymer.

66. The method of claim 64, wherein the coating material comprises alginic acid or an alginate.

67. The method of claim 61, further comprising a pharmaceutically acceptable carrier.

68. The method of claim 61, wherein the core component further comprises an aromatic amino acid- or DOPA-decarboxylase inhibitor.


SEQUENCE LISTING (2 sequences)

SEQ ID NO: 1 - DNA, 2149 bp, synthetic construct
  source 1..2149: mol_type = other DNA
gaaggagatatacatatgaaacccgaagatttccgtgcttcaacacagcgccctttcactggggaagaatacctgaagagcctgcaagacggtcgtgaaatttatatttacggggagcgtgtgaaggatgttacgacccatccagcctttcgcaacgccgctgcgtctgtggcgcagttgtatgatgcgttacacaaacctgagatgcaggattcgttgtgctggaacacagacacgggttcgggaggatatactcataaattttttcgcgttgctaagtcggcagacgacctgcgccaacaacgtgatgctattgctgagtggtcacgtctgtcgtacgggtggatgggacgtacacccgattataaagcggcgtttggatgcgcattgggagctaaccctggattctatggacagttcgagcagaatgcccgcaactggtacacacgcattcaagaaactgggttgtattttaatcacgccattgtcaatccgccgatcgatcgccacctgcccacggataaagtaaaagatgtatatattaagttggaaaaagagacagacgcagggatcattgtatcaggcgccaaggtggttgcgaccaattctgccctgacgcactacaacatgatcggctttggatctgctcaagtgatgggtgaaaaccccgattttgcacttatgtttgtagcccccatggacgctgacggggttaaactgattagccgcgcatcgtacgaaatggtcgccggggccacaggcagtccgtacgattatcctttatctagtcgcttcgacgaaaacgacgcgatcttagtgatggataacgtcctgattccttgggagaacgtcctgatctatcgtgatttcgaccgctgccgtcgttggactatggaaggaggcttcgctcgcatgtaccctttgcaagcctgtgtacgccttgctgtcaaacttgatttcatcactgcgcttttgaagaaatcgttagagtgtactgggacgctggagttccgtggtgtccaagccgaccttggcgaggtggtggcttggcgtaatactttctgggcattatccgactccatgtgctcggaagcaaccccctgggtcaatggggcataccttcccgatcacgccgctcttcaaacctatcgcgtacttgcgcctatggcttatgctaagattaaaaatattatcgaacgtaatgtgacttccggcttaatttacttgccctccagcgcgcgcgatctgaataatcctcaaatcgaccagtatttagcgaagtatgttcgcgggagcaacgggatggatcatgtccagcgcatcaaaatccttaagttaatgtgggatgccattggttcagaatttggcgggcgtcatgaactttatgaaattaattactctggctcgcaagacgagatccgtctgcaatgcttgcgccaggcgcaatcctcgggtaatatggataagatgatggctatggtagaccgctgcctgtccgagtacgaccaaaacggatggacggtcccccatctgcataacaacgatgacattaacatgctggataagctgctgaaataacgcagcaggaggttaagatgcagctggacgaacaacgcctgcgctttcgtgacgcaatggcgagtttgagtgcagccgttaatatcattacaacagaaggggacgcaggtcaatgtggaatcactgcaacggccgtgtgctcagttacagacactccgccttcattaatggtatgcatcaatgctaactcggctatgaatcctgtcttccaaggcaatgggaaattatgtgtgaacgtcctgaaccatgagcaagaactgatggcacgccacttcgcaggcatgacaggtatggcaatggaggagcgttttagcttgtcttgctggcaaaagggaccactggcccaaccagttttgaaggggtctcttgcatcattagaaggggaaatccgcgatgtccaggcgattggtacacacctggtttaccttgtcgagatcaagaacatcattttatccgcagagggccacgggctgatttactttaaacgccgcttccatccggttatgctggaaatggaagctgcaatttaagtaaggaaacatttatgcgcctgcatcatcaccaccatcac

SEQ ID NO: 2 - DNA, 119 bp, Escherichia coli
  source 1..119: mol_type = genomic DNA
caccacaattcagcaaattgtgaacatcatcacgttcatctttccctggttgccaatggcccattttcctgtcagtaacgagaaggtcgcgaattcaggcgctttttagactggtcgta